SEQUENCE LISTING (155 sequences)

SEQ ID NO: 1 - protein, 23 aa, synthetic construct
DIQMTQSPSSLSASVGDRVTITC

SEQ ID NO: 2 - protein, 11 aa, synthetic construct
RASQGISNYLN

SEQ ID NO: 3 - protein, 15 aa, synthetic construct
WYQQKPGKAPKPLIY

SEQ ID NO: 4 - protein, 7 aa, synthetic construct
YTSNLQS

SEQ ID NO: 5 - protein, 32 aa, synthetic construct
GVPSRFSGSGSGTDYTLTISSLQPEDFATYYC

SEQ ID NO: 6 - protein, 9 aa, synthetic construct
MGQTISSYT

SEQ ID NO: 7 - protein, 10 aa, synthetic construct
FGQGTKLEIK

SEQ ID NO: 8 - protein, 30 aa, synthetic construct
EVQLVESGGGLVQPGGSLRLSCAASGFTFS

SEQ ID NO: 9 - protein, 5 aa, synthetic construct
NFDMA

SEQ ID NO: 10 - protein, 14 aa, synthetic construct
WVRQAPGKGLVWVS

SEQ ID NO: 11 - protein, 17 aa, synthetic construct
SITTGADHAIYADSVKG

SEQ ID NO: 12 - protein, 32 aa, synthetic construct
RFTISRDNAKNTLYLQMNSLRAEDTAVYYCVR

SEQ ID NO: 13 - protein, 12 aa, synthetic construct
HGYYDGYHLFDY

SEQ ID NO: 14 - protein, 11 aa, synthetic construct
WGQGTLVTVSS

SEQ ID NO: 15 - protein, 11 aa, synthetic construct
RASQDISKYLN

SEQ ID NO: 16 - protein, 7 aa, synthetic construct
HTSRLHS

SEQ ID NO: 17 - protein, 9 aa, synthetic construct
QQGNTLPYT

SEQ ID NO: 18 - protein, 5 aa, synthetic construct
DYGVS

SEQ ID NO: 19 - protein, 16 aa, synthetic construct
VIWGSETTYYNSALKS

SEQ ID NO: 20 - protein, 12 aa, synthetic construct
HYYYGGSYAMDY

SEQ ID NO: 21 - DNA, 63 bp, synthetic construct
  source 1..63: mol_type = other DNA
atggccttaccagtgaccgccttgctcctgccgctggccttgctgctccacgccgccaggccg

SEQ ID NO: 22 - protein, 21 aa, synthetic construct
MALPVTALLLPLALLLHAARP

SEQ ID NO: 23 - DNA, 321 bp, synthetic construct
  source 1..321: mol_type = other DNA
gacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaag

SEQ ID NO: 24 - protein, 107 aa, synthetic construct
DIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 25 - DNA, 45 bp, synthetic construct
  source 1..45: mol_type = other DNA
ggtggcggtggctcgggcggtggtgggtcgggtggcggcggatct

SEQ ID NO: 26 - protein, 15 aa, synthetic construct
GGGGSGGGGSGGGGS

SEQ ID NO: 27 - DNA, 363 bp, synthetic construct
  source 1..363: mol_type = other DNA
gaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctcc

SEQ ID NO: 28 - protein, 121 aa, synthetic construct
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSS

SEQ ID NO: 29 - DNA, 15 bp, synthetic construct
  source 1..15: mol_type = other DNA
ggaggtggtggatcc

SEQ ID NO: 30 - protein, 5 aa, synthetic construct
GGGGS

SEQ ID NO: 31 - DNA, 321 bp, synthetic construct
  source 1..321: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcaca

SEQ ID NO: 32 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 33 - DNA, 45 bp, synthetic construct; identical to SEQ ID NO: 25
  source 1..45: mol_type = other DNA

SEQ ID NO: 34 - protein, 15 aa, synthetic construct
GGGGSGGGGSGGGGS

SEQ ID NO: 35 - DNA, 360 bp, synthetic construct
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 36 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - DNA, 36 bp, synthetic construct
  source 1..36: mol_type = other DNA
gagagcaagtacggaccgccctgccccccttgccct

SEQ ID NO: 38 - protein, 12 aa, synthetic construct
ESKYGPPCPPCP

SEQ ID NO: 39 - DNA, 84 bp, synthetic construct
  source 1..84: mol_type = other DNA
atgttctgggtgctggtggtggtcggaggcgtgctggcctgctacagcctgctggtcaccgtggccttcatcatcttttgggtg

SEQ ID NO: 40 - protein, 28 aa, synthetic construct
MFWVLVVVGGVLACYSLLVTVAFIIFWV

SEQ ID NO: 41 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 42 - protein, 42 aa, synthetic construct
KRGRKKLLYIFKQPFMRPVQTTQEEDGCSCRFPEEEEGGCEL

SEQ ID NO: 43 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 44 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 2118 bp, synthetic construct
  source 1..2118: mol_type = other DNA
atggccttaccagtgaccgccttgctcctgccgctggccttgctgctccacgccgccaggccggacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggtggcggtggctcgggcggtggtgggtcgggtggcggcggatctgaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggaggtggtggatccgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtaa

SEQ ID NO: 46 - protein, 705 aa, synthetic construct
MALPVTALLLPLALLLHAARPDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 63 bp, synthetic construct; identical to SEQ ID NO: 21
  source 1..63: mol_type = other DNA

SEQ ID NO: 48 - DNA, 363 bp, synthetic construct; identical to SEQ ID NO: 27
  source 1..363: mol_type = other DNA

SEQ ID NO: 49 - DNA, 45 bp, synthetic construct; identical to SEQ ID NO: 25
  source 1..45: mol_type = other DNA

SEQ ID NO: 50 - DNA, 321 bp, synthetic construct; identical to SEQ ID NO: 23
  source 1..321: mol_type = other DNA

SEQ ID NO: 51 - DNA, 15 bp, synthetic construct
  source 1..15: mol_type = other DNA
ggaggtggtggatcc

SEQ ID NO: 52 - DNA, 321 bp, synthetic construct; identical to SEQ ID NO: 31
  source 1..321: mol_type = other DNA

SEQ ID NO: 53 - DNA, 45 bp, synthetic construct; identical to SEQ ID NO: 25
  source 1..45: mol_type = other DNA

SEQ ID NO: 54 - DNA, 360 bp, synthetic construct; identical to SEQ ID NO: 35
  source 1..360: mol_type = other DNA

SEQ ID NO: 55 - DNA, 36 bp, synthetic construct; identical to SEQ ID NO: 37
  source 1..36: mol_type = other DNA

SEQ ID NO: 56 - DNA, 84 bp, synthetic construct; identical to SEQ ID NO: 39
  source 1..84: mol_type = other DNA

SEQ ID NO: 57 - DNA, 126 bp, synthetic construct; identical to SEQ ID NO: 41
  source 1..126: mol_type = other DNA

SEQ ID NO: 58 - DNA, 336 bp, synthetic construct
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 59 - DNA, 2118 bp, synthetic construct
  source 1..2118: mol_type = other DNA
atggccttaccagtgaccgccttgctcctgccgctggccttgctgctccacgccgccaggccggaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggtggcggtggctcgggcggtggtgggtcgggtggcggcggatctgacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggaggtggtggatccgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtaa

SEQ ID NO: 60 - protein, 705 aa, synthetic construct
MALPVTALLLPLALLLHAARPEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 63 bp, synthetic construct; identical to SEQ ID NO: 21
  source 1..63: mol_type = other DNA

SEQ ID NO: 62 - DNA, 363 bp, synthetic construct; identical to SEQ ID NO: 27
  source 1..363: mol_type = other DNA

SEQ ID NO: 63 - DNA, 45 bp, synthetic construct; identical to SEQ ID NO: 25
  source 1..45: mol_type = other DNA

SEQ ID NO: 64 - DNA, 321 bp, synthetic construct; identical to SEQ ID NO: 23
  source 1..321: mol_type = other DNA

SEQ ID NO: 65 - DNA, 15 bp, synthetic construct
  source 1..15: mol_type = other DNA
ggaggtggtggatcc

SEQ ID NO: 66 - DNA, 360 bp, synthetic construct; identical to SEQ ID NO: 35
  source 1..360: mol_type = other DNA

SEQ ID NO: 67 - DNA, 45 bp, synthetic construct; identical to SEQ ID NO: 25
  source 1..45: mol_type = other DNA

SEQ ID NO: 68 - DNA, 321 bp, synthetic construct; identical to SEQ ID NO: 31
  source 1..321: mol_type = other DNA

SEQ ID NO: 69 - DNA, 36 bp, synthetic construct; identical to SEQ ID NO: 37
  source 1..36: mol_type = other DNA

SEQ ID NO: 70 - DNA, 84 bp, synthetic construct; identical to SEQ ID NO: 39
  source 1..84: mol_type = other DNA

SEQ ID NO: 71 - DNA, 126 bp, synthetic construct; identical to SEQ ID NO: 41
  source 1..126: mol_type = other DNA

SEQ ID NO: 72 - DNA, 336 bp, synthetic construct; identical to SEQ ID NO: 58
  source 1..336: mol_type = other DNA

SEQ ID NO: 73 - DNA, 2118 bp, synthetic construct
  source 1..2118: mol_type = other DNA
atggccttaccagtgaccgccttgctcctgccgctggccttgctgctccacgccgccaggccggaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggtggcggtggctcgggcggtggtgggtcgggtggcggcggatctgacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggaggtggtggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggtggcggtggctcgggcggtggtgggtcgggtggcggcggatctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacagagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtaa

SEQ ID NO: 74 - protein, 705 aa, synthetic construct
MALPVTALLLPLALLLHAARPEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 63 bp, synthetic construct; identical to SEQ ID NO: 21
  source 1..63: mol_type = other DNA

SEQ ID NO: 76 - DNA, 321 bp, synthetic construct; identical to SEQ ID NO: 23
  source 1..321: mol_type = other DNA

SEQ ID NO: 77 - DNA, 45 bp, synthetic construct; identical to SEQ ID NO: 25
  source 1..45: mol_type = other DNA

SEQ ID NO: 78 - DNA, 363 bp, synthetic construct; identical to SEQ ID NO: 27
  source 1..363: mol_type = other DNA

SEQ ID NO: 79 - DNA, 15 bp, synthetic construct
  source 1..15: mol_type = other DNA
ggaggtggtggatcc

SEQ ID NO: 80 - DNA, 360 bp, synthetic construct; identical to SEQ ID NO: 35
  source 1..360: mol_type = other DNA

SEQ ID NO: 81 - DNA, 45 bp, synthetic construct; identical to SEQ ID NO: 25
  source 1..45: mol_type = other DNA

SEQ ID NO: 82 - DNA, 321 bp, synthetic construct; identical to SEQ ID NO: 31
  source 1..321: mol_type = other DNA

SEQ ID NO: 83 - DNA, 36 bp, synthetic construct; identical to SEQ ID NO: 37
  source 1..36: mol_type = other DNA

SEQ ID NO: 84 - DNA, 84 bp, synthetic construct; identical to SEQ ID NO: 39
  source 1..84: mol_type = other DNA

SEQ ID NO: 85 - DNA, 126 bp, synthetic construct; identical to SEQ ID NO: 41
  source 1..126: mol_type = other DNA

SEQ ID NO: 86 - DNA, 336 bp, synthetic construct; identical to SEQ ID NO: 58
  source 1..336: mol_type = other DNA

SEQ ID NO: 87 - DNA, 2118 bp, synthetic construct
  source 1..2118: mol_type = other DNA
atggccttaccagtgaccgccttgctcctgccgctggccttgctgctccacgccgccaggccggacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggtggcggtggctcgggcggtggtgggtcgggtggcggcggatctgaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggaggtggtggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggtggcggtggctcgggcggtggtgggtcgggtggcggcggatctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacagagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtaa

SEQ ID NO: 88 - protein, 705 aa, synthetic construct
MALPVTALLLPLALLLHAARPDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 63 bp, synthetic construct; identical to SEQ ID NO: 21
  source 1..63: mol_type = other DNA

SEQ ID NO: 90 - DNA, 321 bp, synthetic construct; identical to SEQ ID NO: 23
  source 1..321: mol_type = other DNA

SEQ ID NO: 91 - DNA, 45 bp, synthetic construct; identical to SEQ ID NO: 25
  source 1..45: mol_type = other DNA

SEQ ID NO: 92 - DNA, 363 bp, synthetic construct; identical to SEQ ID NO: 27
  source 1..363: mol_type = other DNA

SEQ ID NO: 93 - DNA, 15 bp, synthetic construct
  source 1..15: mol_type = other DNA
ggaggtggtggatcc

SEQ ID NO: 94 - DNA, 321 bp, synthetic construct; identical to SEQ ID NO: 31
  source 1..321: mol_type = other DNA

SEQ ID NO: 95 - DNA, 45 bp, synthetic construct; identical to SEQ ID NO: 25
  source 1..45: mol_type = other DNA

SEQ ID NO: 96 - DNA, 360 bp, synthetic construct; identical to SEQ ID NO: 35
  source 1..360: mol_type = other DNA

SEQ ID NO: 97 - DNA, 165 bp, synthetic construct
  source 1..165: mol_type = other DNA
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 98 - protein, 55 aa, synthetic construct
FVPVFLPAKPTTTPAPRPPTPAPTIASQPLSLRPEACRPAAGGAVHTRGLDFACD

SEQ ID NO: 99 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 100 - protein, 24 aa, synthetic construct
IYIWAPLAGTCGVLLLSLVITLYC

SEQ ID NO: 101 - DNA, 126 bp, synthetic construct; identical to SEQ ID NO: 41
  source 1..126: mol_type = other DNA

SEQ ID NO: 102 - DNA, 339 bp, synthetic construct; identical to SEQ ID NO: 43
  source 1..339: mol_type = other DNA

SEQ ID NO: 103 - DNA, 2235 bp, synthetic construct
  source 1..2235: mol_type = other DNA
atggccttaccagtgaccgccttgctcctgccgctggccttgctgctccacgccgccaggccggacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggtggcggtggctcgggcggtggtgggtcgggtggcggcggatctgaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggaggtggtggatccgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 104 - protein, 744 aa, synthetic construct
MALPVTALLLPLALLLHAARPDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - DNA, 63 bp, synthetic construct; identical to SEQ ID NO: 21
  source 1..63: mol_type = other DNA

SEQ ID NO: 106 - DNA, 363 bp, synthetic construct; identical to SEQ ID NO: 27
  source 1..363: mol_type = other DNA

SEQ ID NO: 107 - DNA, 45 bp, synthetic construct; identical to SEQ ID NO: 25
  source 1..45: mol_type = other DNA

SEQ ID NO: 108 - protein, 15 aa, synthetic construct
GGGGSGGGGSGGGGS

SEQ ID NO: 109 - DNA, 321 bp, synthetic construct; identical to SEQ ID NO: 23
  source 1..321: mol_type = other DNA

SEQ ID NO: 110 - DNA, 15 bp, synthetic construct
  source 1..15: mol_type = other DNA
ggaggtggtggatcc

SEQ ID NO: 111 - protein, 5 aa, synthetic construct
GGGGS

SEQ ID NO: 112 - DNA, 321 bp, synthetic construct; identical to SEQ ID NO: 31
  source 1..321: mol_type = other DNA

SEQ ID NO: 113 - DNA, 45 bp, synthetic construct; identical to SEQ ID NO: 25
  source 1..45: mol_type = other DNA

SEQ ID NO: 114 - protein, 15 aa, synthetic construct
GGGGSGGGGSGGGGS

SEQ ID NO: 115 - DNA, 360 bp, synthetic construct; identical to SEQ ID NO: 35
  source 1..360: mol_type = other DNA

SEQ ID NO: 116 - DNA, 165 bp, synthetic construct; identical to SEQ ID NO: 97
  source 1..165: mol_type = other DNA

SEQ ID NO: 117 - DNA, 72 bp, synthetic construct; identical to SEQ ID NO: 99
  source 1..72: mol_type = other DNA

SEQ ID NO: 118 - DNA, 126 bp, synthetic construct; identical to SEQ ID NO: 41
  source 1..126: mol_type = other DNA

SEQ ID NO: 119 - DNA, 339 bp, synthetic construct; identical to SEQ ID NO: 43
  source 1..339: mol_type = other DNA

SEQ ID NO: 120 - DNA, 2235 bp, synthetic construct
  source 1..2235: mol_type = other DNA
atggccttaccagtgaccgccttgctcctgccgctggccttgctgctccacgccgccaggccggaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggtggcggtggctcgggcggtggtgggtcgggtggcggcggatctgacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggaggtggtggatccgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 121 - protein, 744 aa, synthetic construct
MALPVTALLLPLALLLHAARPEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - DNA, 63 bp, synthetic construct; identical to SEQ ID NO: 21
  source 1..63: mol_type = other DNA

SEQ ID NO: 123 - DNA, 363 bp, synthetic construct; identical to SEQ ID NO: 27
  source 1..363: mol_type = other DNA

SEQ ID NO: 124 - DNA, 45 bp, synthetic construct; identical to SEQ ID NO: 25
  source 1..45: mol_type = other DNA

SEQ ID NO: 125 - protein, 15 aa, synthetic construct
GGGGSGGGGSGGGGS

SEQ ID NO: 126 - DNA, 321 bp, synthetic construct; identical to SEQ ID NO: 23
  source 1..321: mol_type = other DNA

SEQ ID NO: 127 - DNA, 15 bp, synthetic construct
  source 1..15: mol_type = other DNA
ggaggtggtggatcc

SEQ ID NO: 128 - protein, 5 aa, synthetic construct
GGGGS

SEQ ID NO: 129 - DNA, 360 bp, synthetic construct; identical to SEQ ID NO: 35
  source 1..360: mol_type = other DNA

SEQ ID NO: 130 - DNA, 45 bp, synthetic construct; identical to SEQ ID NO: 25
  source 1..45: mol_type = other DNA

SEQ ID NO: 131 - protein, 15 aa, synthetic construct
GGGGSGGGGSGGGGS

SEQ ID NO: 132 - DNA, 321 bp, synthetic construct; identical to SEQ ID NO: 31
  source 1..321: mol_type = other DNA

SEQ ID NO: 133 - DNA, 165 bp, synthetic construct; identical to SEQ ID NO: 97
  source 1..165: mol_type = other DNA

SEQ ID NO: 134 - DNA, 72 bp, synthetic construct; identical to SEQ ID NO: 99
  source 1..72: mol_type = other DNA

SEQ ID NO: 135 - DNA, 126 bp, synthetic construct; identical to SEQ ID NO: 41
  source 1..126: mol_type = other DNA

SEQ ID NO: 136 - DNA, 339 bp, synthetic construct; identical to SEQ ID NO: 43
  source 1..339: mol_type = other DNA

SEQ ID NO: 137 - DNA, 2235 bp, synthetic construct
  source 1..2235: mol_type = other DNA
atggccttaccagtgaccgccttgctcctgccgctggccttgctgctccacgccgccaggccggaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggtggcggtggctcgggcggtggtgggtcgggtggcggcggatctgacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggaggtggtggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggtggcggtggctcgggcggtggtgggtcgggtggcggcggatctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 138 - protein, 744 aa, synthetic construct
MALPVTALLLPLALLLHAARPEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - DNA, 63 bp, synthetic construct; identical to SEQ ID NO: 21
  source 1..63: mol_type = other DNA

SEQ ID NO: 140 - DNA, 321 bp, synthetic construct; identical to SEQ ID NO: 23
  source 1..321: mol_type = other DNA

SEQ ID NO: 141 - DNA, 45 bp, synthetic construct; identical to SEQ ID NO: 25
  source 1..45: mol_type = other DNA

SEQ ID NO: 142 - protein, 15 aa, synthetic construct
GGGGSGGGGSGGGGS

SEQ ID NO: 143 - DNA, 363 bp, synthetic construct; identical to SEQ ID NO: 27
  source 1..363: mol_type = other DNA

SEQ ID NO: 144 - DNA, 15 bp, synthetic construct
  source 1..15: mol_type = other DNA
ggaggtggtggatcc

SEQ ID NO: 145 - protein, 5 aa, synthetic construct
GGGGS

SEQ ID NO: 146 - DNA, 360 bp, synthetic construct; identical to SEQ ID NO: 35
  source 1..360: mol_type = other DNA

SEQ ID NO: 147 - DNA, 45 bp, synthetic construct; identical to SEQ ID NO: 25
  source 1..45: mol_type = other DNA

SEQ ID NO: 148 - protein, 15 aa, synthetic construct
GGGGSGGGGSGGGGS

SEQ ID NO: 149 - DNA, 321 bp, synthetic construct; identical to SEQ ID NO: 31
  source 1..321: mol_type = other DNA

SEQ ID NO: 150 - DNA, 165 bp, synthetic construct; identical to SEQ ID NO: 97
  source 1..165: mol_type = other DNA

SEQ ID NO: 151 - DNA, 72 bp, synthetic construct; identical to SEQ ID NO: 99
  source 1..72: mol_type = other DNA

SEQ ID NO: 152 - DNA, 126 bp, synthetic construct; identical to SEQ ID NO: 41
  source 1..126: mol_type = other DNA

SEQ ID NO: 153 - DNA, 339 bp, synthetic construct; identical to SEQ ID NO: 43
  source 1..339: mol_type = other DNA

SEQ ID NO: 154 - DNA, 2235 bp, synthetic construct
  source 1..2235: mol_type = other DNA
atggccttaccagtgaccgccttgctcctgccgctggccttgctgctccacgccgccaggccggacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggtggcggtggctcgggcggtggtgggtcgggtggcggcggatctgaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccggaggtggtggatccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggtggcggtggctcgggcggtggtgggtcgggtggcggcggatctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 155 - protein, 744 aa, synthetic construct
MALPVTALLLPLALLLHAARPDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0027156 A1
(43) Pub. Date: Jan. 29, 2026

(54) BISPECIFIC CHIMERIC ANTIGEN RECEPTORS TARGETING BCMA AND CD19

(71) Applicant: ABELZETA INC., Rockville, MD (US)

(72) Inventors: Yihong YAO, Rockville, MD (US); Jiaqi HUANG, Rockville, MD (US); Shigui ZHU, Rockville, MD (US); Xin YAO, Rockville, MD (US); Xiaobing LUO, Rockville, MD (US); Yanfeng LI, Rockville, MD (US)

(73) Assignee: ABELZETA INC., Rockville, MD (US)

(21) Appl. No.: 19/351,823

(22) Filed: Oct. 7, 2025

Related U.S. Application Data
(63) Continuation of application No. PCT/US2024/023797, filed on Apr. 10, 2024.
(60) Provisional application No. 63/495,377, filed on Apr. 11, 2023.

(51) Int. Cl. A61K 35/17; A61K 40/11; A61K 40/31; A61K 40/42; A61P 35/00; C07K 14/725; C07K 16/28; C12N 5/0783

(52) U.S. Cl. CPC A61K 35/17; A61K 40/11; A61K 40/31; A61K 40/4211; A61K 40/4215; A61P 35/00; C07K 14/7051; C07K 16/2803; C07K 16/2878; C12N 5/0636; A61K 2239/13; A61K 2239/17; A61K 2239/21; C07K 2317/31; C07K 2317/53; C07K 2317/565; C07K 2317/622; C07K 2319/02; C07K 2319/03

(57) ABSTRACT

The present disclosure provides bispecific chimeric antigen receptors targeting BCMA and CD19. The CAR may comprise an scFv targeting BCMA and an scFv targeting CD19, a hinge region, a transmembrane domain, a co-stimulatory region, and a cytoplasmic signaling domain. The chimeric antigen receptors can be used to treat autoimmune disorders or cancer.

### CROSS REFERENCE TO RELATED APPLICATIONS

The present application is continuation of International Patent Application No. PCT/US2024/023797 filed on Apr. 10, 2024, which claims priority to U.S. Provisional Patent Application No. 63/495,377, filed on Apr. 11, 2023, all of which are hereby incorporated by reference in their entireties.

### INCORPORATION-BY-REFERENCE OF SEQUENCE LISTING

A sequence listing, filed as the XML file “Sequence Listing_11299_011843-US1.xml” which was created on Oct. 7, 2025 and is 175,878 bytes in size, is incorporated herein by reference in its entirety.

### FIELD OF THE INVENTION

The present disclosure relates to the field of immunotherapy, and more particularly to bispecific chimeric antigen receptors (CARs) targeting BCMA and CD19.

### BACKGROUND

Autoimmune diseases are conditions caused by the immune system's response to the body itself, resulting in damage to its own tissues. These are typically divided into two main categories: systemic autoimmune diseases, such as systemic lupus erythematosus (SLE), rheumatoid arthritis, and systemic vasculitis; and organ-specific autoimmune diseases, such as autoimmune hepatitis and type I diabetes. Most autoimmune diseases are difficult to cure and often require long-term or lifelong medication. Treatment primarily involves corticosteroids and immunosuppressants, greatly impacting the patient's quality of life and presenting a significant unmet clinical need (Wang et al., Human autoimmune diseases: a comprehensive update, J. Intern. Med. 2015, 278(4):369-95).

The etiology of autoimmune diseases is unclear. In patients, abnormal activation of humoral immunity occurs, leading to the production of a large number of antibodies against self-antigens. These combine to form pathogenic immune complexes, which then deposit locally and cause inflammatory reactions. B cells play an important role in the pathogenesis of autoimmune diseases, promoting the occurrence of autoimmune diseases through various mechanisms such as producing autoantibodies, releasing cytokines, and presenting autoantigens. Autoantibodies, as a key factor, can bind with autoantigens to form immune complexes, which can activate innate immune system cells to produce type I interferon and other pro-inflammatory cytokines resulting in organ damage. Therefore, the depletion or removal of lymphocytes has become a potential treatment strategy.

SLE is a prototypic autoimmune disease that is known to be associated with polyclonal B-cell hyperreactivity (Dorner et al., Mechanisms of B cell autoimmunity in SLE, Arthritis Res. Ther. 13, 243 (2011)). As such, one of the immunological hallmarks of SLE is the production of antinuclear antibodies (ANAs), which can mediate SLE pathogenesis by binding to respective autoantigens, resulting in deposition of immune complexes and induction of inflammation and organ damage (for example, lupus nephritis) (Salmon, J. E., Arming T cells against B cells in systemic lupus erythematosus, Nat. Med. 28, 2009-2010 (2022)). There are two main types of ANAs: anti-DNA antibodies and antibodies recognizing RNA-binding proteins (RBP) (Pisetsky et al., New insights into the role of antinuclear antibodies in systemic lupus erythematosus, Nat. Rev. Rheumatol. 16, 565-579 (2020)). In patients with SLE, the sources of autoantibodies include not only B cells but also a subset of plasma cells termed long-lived plasma cells (LLPCs). While the anti-DNA antibodies are produced by naïve B cells that transition to memory B cells and plasmablasts, which maintain high level expression of CD19 and CD20 on the cell surface, the anti-RBP antibodies are produced by LLPCs, which may lose surface expression of CD19 and CD20, but are positive for B-cell maturation antigen (BCMA), a cell surface protein expressed on all mature plasma cells (Dogan et al., B-cell maturation antigen expression across hematologic cancers: a systematic literature review. Blood Cancer J. 10, 73 (2020); Morgan et al., Unraveling B cell trajectories at single cell resolution, Trends Immunol. 43, 210-229 (2022)). Recent studies demonstrated that a CD11chiT-bet⁺ B cell subset is expanded in human SLE and serves as precursors of autoantibody producing plasma cells. This B cell subset displays high expression of CD19 and CD20 and corresponds to the autoreactive, murine age-associated B cells (autoreactive B cells or ABCs; the term may be used to represent human CD11c^{hi}T-bet⁺ B cells) (Jenks et al., Distinct Effector B Cells Induced by Unregulated Toll-like Receptor 7 Contribute to Pathogenic Responses in Systemic Lupus Erythematosus, Immunity 49, 725-739 e726 (2018); Wang et al., IL-21 drives expansion and plasma cell differentiation of autoreactive CD11c(hi)T-bet(+) B cells in SLE, Nat. Commun. 9, 1758 (2018)). In addition to autoantibody production, B cells also participate in the pathogenesis of SLE and other autoimmune diseases by secreting cytokines and acting as antigen-presenting cells. Therefore, depleting B cells in patients with SLE can be an effective therapy for this life-threatening disease.

B cell depletion could be achieved by administration of monoclonal antibodies against B cell surface markers. Although the anti-CD20 antibody rituximab was successful in early open-label trials in SLE, it failed to meet its primary end points in two randomized controlled trials (Lee et al., B cell depletion therapies in autoimmune disease: advances and mechanistic insights, Nat. Rev. Drug Discov. 20, 179-199 (2021)). Other antibodies targeting CD19 (obexelimab) were also tested in SLE. Although patients receiving obexelimab sustained their level of disease inactivity despite steroid withdrawal in initial studies, phase II clinical trials, failed to meet their primary end points (Lee et al., B cell depletion therapies in autoimmune disease: advances and mechanistic insights, Nat. Rev. Drug Discov. 20, 179-199 (2021)).

One promising approach to achieve B cell depletion is adoptive transfer of CAR-T cells. CAR-T cells are genetically engineered T lymphocytes that, in the absence of major histocompatibility complex (MHC), can recognize specific antigens on target cells, proliferate, and generate cytotoxic immune responses. In a recent study, compassionate-use of CD19 CAR-T therapy in 5 patients with refractory SLE led to deep depletion of B cells and drug-free remission, suggesting that CAR-T cell transfer is feasible, tolerable, and highly effective in SLE (Mackensen et al., Anti-CD19 CAR T cell therapy for refractory systemic lupus erythematosus, Nat. Med. 28, 2124-2132 (2022)).

There is still an urgent need to develop methods to effectively treat autoimmune diseases.

### SUMMARY

The present disclosure provides for a bispecific chimeric antigen receptor (CAR), comprising: (i) an anti-BCMA antigen-binding region which comprises a light chain variable region (V_{L}1) and a heavy chain variable region (V_{H}1), wherein V_{L}1 comprises three complementarity determining regions (CDRs), CDR1, CDR2 and CDR3, having amino acid sequences about 80% to about 100% identical to the amino acid sequences set forth in SEQ ID NO: 2, SEQ ID NO: 4, SEQ ID NO: 6, respectively, and wherein VHl Comprises three CDRs, CDR1, CDR2 and CDR3, having amino acid sequences about 80% to about 100% identical to the amino acid sequences set forth in SEQ ID NO: 9, SEQ ID NO: 11, SEQ ID NO: 13, respectively; and (ii) an anti-CD19 antigen-binding region which comprises a light chain variable region (V_{L}2) and a heavy chain variable region (V_{H}2), wherein V_{L}2 comprises three complementarity determining regions (CDRs), CDR1, CDR2 and CDR3, having amino acid sequences about 80% to about 100% identical to the amino acid sequences set forth in SEQ ID NO: 15, SEQ ID NO: 16, SEQ ID NO: 17, respectively, and wherein V_{H}2 comprises three CDRs, CDR1, CDR2 and CDR3, having amino acid sequences about 80% to about 100% identical to the amino acid sequences set forth in SEQ ID NO: 18, SEQ ID NO: 19, SEQ ID NO: 20, respectively.

The present disclosure provides for a bispecific chimeric antigen receptor (CAR), comprising: (i) an anti-BCMA antigen-binding region which comprises a light chain variable region (V_{L}1) and a heavy chain variable region (V_{H}1); and (ii) an anti-CD19 antigen-binding region which comprises a light chain variable region (V_{L}2) and a heavy chain variable region (V_{H}2).

In one embodiment, V_{L}1 is located at the N-terminus of V_{H}1. In one embodiment, V_{H}1 is located at the N-terminus of V_{L}1. In one embodiment, V_{L}2 is located at the N-terminus of V_{H}2. In one embodiment, V_{H}2 is located at the N-terminus of V_{L}2.

In certain embodiments, V_{L}1 and V_{H}1 have amino acid sequences about 80% to about 100% identical to amino acid sequences set forth in SEQ ID NO: 24 and SEQ ID NO: 28, respectively.

In certain embodiments, V_{L}2 and V_{H}2 have amino acid sequences about 80% to about 100% identical to amino acid sequences set forth in SEQ ID NO: 32 and SEQ ID NO: 36, respectively.

The anti-BCMA antigen-binding region may be a single-chain variable fragment (scFv) that specifically binds BCMA. The anti-CD19 antigen-binding region may be a scFv that specifically binds CD19.

The bispecific CAR may further comprise one or more of the following: (a) a signal peptide, (b) a hinge region, (c) a transmembrane domain, (d) a co-stimulatory region, and (e) a cytoplasmic signaling domain.

The hinge region may comprise a hinge region of IgG4, CD8, CD28, CD137, or combinations thereof.

The transmembrane domain may comprise a transmembrane domain of CD8, CD28, CD3ε, CD45, CD4, CD5, CD9, CD16, CD22, CD33, CD37, CD64, CD80, CD86, CD134, CD137, CD154, or combinations thereof.

The co-stimulatory region may comprise a co-stimulatory region of 4-1BB (CD137), CD28, OX40, CD2, CD7, CD27, CD30, CD40, CD70, CD134, PD1, Dap10, CDS, ICAM-1, LFA-1 (CD11a/CD18), ICOS (CD278), NKG2D, GITR, TLR2, or combinations thereof.

The cytoplasmic signaling domain may comprise a cytoplasmic signaling domain of CD3ζ.

The present disclosure provides for a bispecific CAR comprising (or having) an amino acid sequence about 80% to about 100% identical to the amino acid sequence set forth in SEQ ID NO:46, SEQ ID NO:60, SEQ ID NO:74, SEQ ID NO:88, SEQ ID NO:104, SEQ ID NO:121, SEQ ID NO:138, or SEQ ID NO:155.

Also encompassed by the present disclosure is an immune cell expressing the bispecific CAR. The immune cell may be a T cell or a natural killer (NK) cell.

The present disclosure provides for a nucleic acid encoding the bispecific CAR.

The present disclosure provides for a vector comprising the present nucleic acid encoding the bispecific CAR.

The present disclosure provides for a pharmaceutical composition comprising the bispecific CAR, the immune cell, the nucleic acid, or the vector.

The present disclosure also provides for a method of treating an autoimmune disorder. The method may comprise administering the immune cell, or the pharmaceutical composition, to a subject in need thereof.

The autoimmune disorder may be systemic lupus erythematosus (SLE) (e.g., lupus nephritis), systemic vasculitis, systemic sclerosis, inflammatory myopathy (e.g., polymyositis, dermatomyositis, or inclusion-body myositis), systemic scleroderma, multiple sclerosis, myasthenia gravis, a myositis autoantibody-driven disease, or neuromyelitis optica.

The autoimmune disorder may be polymyositis, dermatomyositis, or inclusion-body myositis. The autoimmune disorder may be lupus nephritis.

The present disclosure also provides for a method of treating cancer. The method may comprise administering the immune cell, or the pharmaceutical composition, to a subject in need thereof.

The cancer may be a hematologic cancer. The cancer may be a B-cell malignancy. The cancer may be Hodgkin's lymphoma, non-Hodgkin's lymphoma, leukemia, and/or multiple myeloma. The cancer may be acute myeloid leukemia (AML), multiple myeloma (MM), chronic lymphocytic leukemia (CLL), chronic myelogenous leukemia, acute lymphoblastic leukemia (ALL), diffuse large B cell lymphoma (DLBCL), or combinations thereof.

The immune cell may be allogeneic or autologous.

### BRIEF DESCRIPTION OF THE DRAWINGS

FIG. 1 shows the structures of the bispecific chimeric antigen receptors targeting CD19 and BCMA. The structures of the CAR include a signal peptide (SP), an anti-BCMA scFv (B20), a linker (linker-2), an anti-CD19 scFv (FMC63), a hinge region, a transmembrane domain, a co-stimulatory region, and a cytoplasmic signaling domain (CD3ζ). A short IgG4 hinge (12 aa) and a CD28 transmembrane domain are included in TB19-1 to TB19-4; a CD8a hinge (55 aa) and a CD8a transmembrane domain are included in TB19-L1 to TB19-L4. Four combinations of orientations of V_{H} and V_{L} in the two scFv sequences are included in the two groups of CARs (TB19-1 to 4 and TB19-L1 to L4).

FIGS. 2A-2B show the expression level of anti-BCMA (FIG. 2A) and anti-CD19 (FIG. 2B) CARs on the surface of T cells. C-CAR088 is an anti-BCMA CAR. C-CAR011 is an anti-CD19 CAR.

FIGS. 3A-3B show the levels of IFN-7 secreted by the activated anti-BCMA/CD19 CAR-T cells in vitro in the cell culture supernatant. FIG. 3A shows the levels of IFN-γ secreted by the TB19-1 to TB19-4 and TB19-L1 to TB19-L4 CAR-T cells in the cell culture supernatant.

FIG. 3B shows high IFN-γ release by the TB19-1 to TB19-4 CAR-T cells. MM.1S is a BCMA-positive MM cell line; RAJI is CD19 positive and BCMA positive.

FIGS. 4A-4B show the expression levels of CD137 on the surface of the activated CAR-T cells. FIG. 4A: TB19-1 to TB19-4; FIG. 4B: TB19-L1 to TB19-L4.

FIG. 5 shows the in vitro cytotoxicity of CAR-Ts cells by RTCA assays.

### DETAILED DESCRIPTION

The present disclosure provides a chimeric antigen receptor (CAR) that targets both CD19 and BCMA. The CAR may comprise a signal peptide, an anti-CD19 scFv, an anti-BCMA scFv, a hinge region, a transmembrane domain, a co-stimulatory region, and a cytoplasmic signaling domain. The present CARs can be used to treat autoimmune diseases or cancer.

The present disclosure provides for a bispecific chimeric antigen receptor (CAR). The bispecific CAR may comprise: (i) an anti-BCMA antigen-binding region which comprises a light chain variable region (V_{L}1) and a heavy chain variable region (V_{H}1); and (ii) an anti-CD19 antigen-binding region which comprises a light chain variable region (V_{L}2) and a heavy chain variable region (V_{H}2).

The present bispecific chimeric antigen receptor (CAR) may comprise: (i) an anti-BCMA antigen-binding region which comprises a light chain variable region (V_{L}1) and a heavy chain variable region (V_{H}1) having amino acid sequences about 80% to about 100%, about 85% to about 100%, about 90% to about 100%, or about 95% to about 100%, identical to the amino acid sequences set forth in SEQ ID NO: 24 and SEQ ID NO: 28, respectively; and (ii) an anti-CD19 antigen-binding region which comprises a light chain variable region (V_{L}2) and a heavy chain variable region (V_{H}2) having amino acid sequences about 80% to about 100%, about 85% to about 100%, about 90% to about 100%, or about 95% to about 100%, identical to the amino acid sequences set forth in SEQ ID NO: 32 and SEQ ID NO: 36, respectively.

The present disclosure provides for a bispecific chimeric antigen receptor (CAR). The bispecific CAR may comprise: (i) an anti-BCMA antigen-binding region which comprises a light chain variable region (V_{L}1) and a heavy chain variable region (V_{H}1), and (ii) an anti-CD19 antigen-binding region which comprises a light chain variable region (V_{L}2) and a heavy chain variable region (V_{H}2). V_{L}1 may comprise three complementarity determining regions (CDRs), CDR1, CDR2 and CDR3, having amino acid sequences about 80% to about 100%, about 85% to about 100%, about 90% to about 100%, or about 95% to about 100%, identical to the amino acid sequences set forth in SEQ ID NO: 2, SEQ ID NO: 4, SEQ ID NO: 6, respectively. V_{H}1 may comprise three CDRs, CDR1, CDR2 and CDR3, having amino acid sequences about 80% to about 100%, about 85% to about 100%, about 90% to about 100%, or about 95% to about 100%, identical to the amino acid sequences set forth in SEQ ID NO: 9, SEQ ID NO: 11, SEQ ID NO: 13, respectively. V_{L}2 may comprise three complementarity determining regions (CDRs), CDR1, CDR2 and CDR3, having amino acid sequences about 80% to about 100%, about 85% to about 100%, about 90% to about 100%, or about 95% to about 100%, identical to the amino acid sequences set forth in SEQ ID NO: 15, SEQ ID NO: 16, SEQ ID NO: 17, respectively. V_{H}2 may comprise three CDRs, CDR1, CDR2 and CDR3, having amino acid sequences about 80% to about 100%, about 85% to about 100%, about 90% to about 100%, or about 95% to about 100%, identical to the amino acid sequences set forth in SEQ ID NO: 18, SEQ ID NO: 19, SEQ ID NO: 20, respectively.

In certain embodiments, V_{L}1 is located at the N-terminus of V_{H}1. In certain embodiments, V_{H}1 is located at the N-terminus of V_{L}1. In certain embodiments, V_{H}2 is located at the N-terminus of V_{L}2. In certain embodiments, V_{L}2 is located at the N-terminus of V_{H}2. In one embodiment, V_{L}1 is located at the N-terminus of V_{H}1; V_{L}2 is located at the N-terminus of V_{H}2.

In certain embodiments, V_{L}1 and V_{H}1 have amino acid sequences about 80% to about 100% identical to amino acid sequences set forth in SEQ ID NO: 24 and SEQ ID NO: 28, respectively.

In certain embodiments, V_{L}2 and V_{H}2 have amino acid sequences about 80% to about 100% identical to amino acid sequences set forth in SEQ ID NO: 32 and SEQ ID NO: 36, respectively.

In certain embodiments, the antigen-binding region that specifically binds BCMA is located at the N-terminus of the antigen-binding region that specifically binds CD19. In certain embodiments, the antigen-binding region that specifically binds CD19 is located at the N-terminus of the antigen-binding region that specifically binds BCMA.

The anti-BCMA antigen-binding region may be a single-chain variable fragment (scFv) that specifically binds BCMA. The anti-CD19 antigen-binding region may be a scFv that specifically binds CD19. In certain embodiments, the scFv that specifically binds BCMA is located at the N-terminus of the scFv that specifically binds CD19. In certain embodiments, the scFv that specifically binds CD19 is located at the N-terminus of the scFv that specifically binds BCMA.

The bispecific CAR may further comprise one or more of the following: (a) a signal peptide or SP (or a leader sequence), (b) a hinge region, (c) a transmembrane domain, (d) a co-stimulatory region, and (e) a cytoplasmic signaling domain.

The present bispecific CARs may comprise, from N-terminus to C-terminus, a signal peptide, an anti-BCMA scFv, an anti-CD19 scFv, a hinge region, a transmembrane domain, and a co-stimulatory region, and a cytoplasmic signaling domain.

The signal peptide may comprise a signal peptide of (or may be derived from) CD8, CD28, GM-CSF, CD4, CD137, or combinations thereof. In one embodiment, the signal peptide is a signal peptide of (or is derived from) CD8.

In one embodiment, the signal peptide comprises an amino acid sequence about 80% to about 100%, about 85% to about 100%, about 90% to about 100%, or about 95% to about 100%, identical to the amino acid sequence set forth in SEQ ID NO: 22.

The hinge region may comprise a hinge region of (or may be derived from) IgG4, CD8, CD28, CD137, or combinations thereof, wildtype or mutants.

In one embodiment, the hinge region is a hinge region of (or is derived from) IgG4. In one embodiment, the hinge region comprises an amino acid sequence about 80% to about 100%, about 85% to about 100%, about 90% to about 100%, or about 95% to about 100%, identical to the amino acid sequence set forth in SEQ ID NO: 38.

In one embodiment, the hinge region is is a hinge region of (or is derived from) CD8a. In one embodiment, the hinge region comprises an amino acid sequence about 80% to about 100%, about 85% to about 100%, about 90% to about 100%, or about 95% to about 100%, identical to the amino acid sequence set forth in SEQ ID NO: 98.

The transmembrane domain may comprise a transmembrane domain of (or may be derived from) CD8, CD28, CD3F, CD45, CD4, CD5, CD9, CD16, CD22, CD33, CD37, CD64, CD80, CD86, CD134, CD137, CD154, or combinations thereof.

In one embodiment, the transmembrane domain is a transmembrane domain of (or is derived from) CD28. In one embodiment, the transmembrane domain comprises an amino acid sequence about 80% to about 100%, about 85% to about 100%, about 90% to about 100%, or about 95% to about 100%, identical to the amino acid sequence set forth in SEQ ID NO: 40.

In one embodiment, the transmembrane domain is a transmembrane domain of (or is derived from) CD8a. In one embodiment, the transmembrane domain comprises an amino acid sequence about 80% to about 100%, about 85% to about 100%, about 90% to about 100%, or about 95% to about 100%, identical to the amino acid sequence set forth in SEQ ID NO: 100.

The co-stimulatory region may comprise a co-stimulatory region of (or may be derived from) 4-1BB (CD137), CD28, OX40, CD2, CD7, CD27, CD30, CD40, CD70, CD134, PD1, Dap10, CDS, ICAM-1, LFA-1 (CD11a/CD18), ICOS (CD278), NKG2D, GITR, TLR2, or combinations thereof.

In one embodiment, the co-stimulatory region is a co-stimulatory region of (or is derived from) 4-1BB. In one embodiment, the co-stimulatory region comprises an amino acid sequence about 80% to about 100%, about 85% to about 100%, about 90% to about 100%, or about 95% to about 100%, identical to the amino acid sequence set forth in SEQ ID NO: 42.

The cytoplasmic signaling domain may comprise a cytoplasmic signaling domain of (or may be derived from) CD3ζ. In one embodiment, the cytoplasmic signaling domain comprises an amino acid sequence about 80% to about 100%, about 85% to about 100%, about 90% to about 100%, or about 95% to about 100%, identical to the amino acid sequence set forth in SEQ ID NO: 44.

The present CAR may comprise a linker (linker-1) between V_{L} and V_{H} of the anti-BCMA antigen-binding region. In one embodiment, the linker (linker-1) comprises an amino acid sequence about 80% to about 100%, about 85% to about 100%, about 90% to about 100%, or about 95% to about 100%, identical to the amino acid sequence set forth in SEQ ID NO:26.

The present CAR may comprise a linker (linker-2) between the anti-BCMA antigen-binding region and the anti-CD19 antigen-binding region. In one embodiment, the linker (linker-2) comprises an amino acid sequence about 80% to about 100%, about 85% to about 100%, about 90% to about 100%, or about 95% to about 100%, identical to the amino acid sequence set forth in SEQ ID NO:30.

The present CAR may comprise a linker (linker-3) between V_{L} and V_{H} of the anti-CD19 antigen-binding region. In one embodiment, the linker (linker-3) comprises an amino acid sequence about 80% to about 100%, about 85% to about 100%, about 90% to about 100%, or about 95% to about 100%, identical to the amino acid sequence set forth in SEQ ID NO:34.

In one embodiment, the bispecific CAR comprises, from N-terminus to C-terminus, (a) an anti-BCMA antigen-binding region with a light chain variable region (V_{L}1) and a heavy chain variable region (V_{H}1) of those of the BCMA-20 antibody, (ii) an anti-CD19 antigen-binding region with a light chain variable region (V_{L}2) and a heavy chain variable region (V_{H}2) of those of the FMC63 antibody, (iii) a hinge region having an amino acid sequence set forth in SEQ ID NO:38, (iv) a transmembrane domain having an amino acid sequence set forth in SEQ ID NO:40, (v) a co-stimulatory region having an amino acid sequence set forth in SEQ ID NO:42, and (vi) a cytoplasmic signaling domain having an amino acid sequence set forth in SEQ ID NO:44.

In one embodiment, the bispecific CAR comprises, from N-terminus to C-terminus, (a) an anti-BCMA antigen-binding region with a light chain variable region (V_{L}1) and a heavy chain variable region (V_{H}1) having amino acid sequences set forth in SEQ ID NO:24 and SEQ ID NO:28, respectively, (ii) an anti-CD19 antigen-binding region with a light chain variable region (V_{L}2) and a heavy chain variable region (V_{H}2) having amino acid sequences set forth in SEQ ID NO:32 and SEQ ID NO:36, respectively, (iii) a hinge region having an amino acid sequence set forth in SEQ ID NO:38, (iv) a transmembrane domain having an amino acid sequence set forth in SEQ ID NO:40, (v) a co-stimulatory region having an amino acid sequence set forth in SEQ ID NO:42, and (vi) a cytoplasmic signaling domain having an amino acid sequence set forth in SEQ ID NO:44.

In one embodiment, the bispecific CAR comprises, from N-terminus to C-terminus, (a) an anti-BCMA antigen-binding region with a light chain variable region (V_{L}1) and a heavy chain variable region (V_{H}1) of those of the BCMA-20 antibody, (ii) an anti-CD19 antigen-binding region with a light chain variable region (V_{L}2) and a heavy chain variable region (V_{H}2) of those of the FMC63 antibody, (iii) a hinge region having an amino acid sequence set forth in SEQ ID NO:98, (iv) a transmembrane domain having an amino acid sequence set forth in SEQ ID NO:100, (v) a co-stimulatory region having an amino acid sequence set forth in SEQ ID NO:42, and (vi) a cytoplasmic signaling domain having an amino acid sequence set forth in SEQ ID NO:44.

In one embodiment, the bispecific CAR comprises, from N-terminus to C-terminus, (a) an anti-BCMA antigen-binding region with a light chain variable region (V_{L}1) and a heavy chain variable region (V_{H}1) having amino acid sequences set forth in SEQ ID NO:24 and SEQ ID NO:28, respectively, (ii) an anti-CD19 antigen-binding region with a light chain variable region (V_{L}2) and a heavy chain variable region (V_{H}2) having amino acid sequences set forth in SEQ ID NO:32 and SEQ ID NO:36, respectively, (iii) a hinge region having an amino acid sequence set forth in SEQ ID NO:98, (iv) a transmembrane domain having an amino acid sequence set forth in SEQ ID NO:100, (v) a co-stimulatory region having an amino acid sequence set forth in SEQ ID NO:42, and (vi) a cytoplasmic signaling domain having an amino acid sequence set forth in SEQ ID NO:44.

In certain embodiments, V_{L}1 is located at the N-terminus of V_{H}1. In certain embodiments, V_{H}1 is located at the N-terminus of V_{L}1. In certain embodiments, V_{H}2 is located at the N-terminus of V_{L}2. In certain embodiments, V_{L}2 is located at the N-terminus of V_{H}2. In one embodiment, V_{L}1 is located at the N-terminus of V_{H}1; V_{L}2 is located at the N-terminus of V_{H}2.

In certain embodiments, the bispecific CAR comprises an amino acid sequence about 80% to about 100%, about 85% to about 100%, about 90% to about 100%, or about 95% to about 100%, identical to the amino acid sequence set forth in SEQ ID NO:46, SEQ ID NO:60, SEQ ID NO:74, SEQ ID NO:88, SEQ ID NO:104, SEQ ID NO:121, SEQ ID NO:138, or SEQ ID NO:155.

In certain embodiments, the bispecific CAR may have an amino acid sequence set forth in SEQ ID NO:46, SEQ ID NO:60, SEQ ID NO:74, SEQ ID NO:88, SEQ ID NO:104, SEQ ID NO:121, SEQ ID NO:138, or SEQ ID NO:155.

The present bispecific CAR may be encoded by a nucleic acid having a nucleotide sequence about 80% to about 100%, about 85% to about 100%, about 90% to about 100%, or about 95% to about 100%, identical to the nucleotide sequence set forth in SEQ ID NO:45, SEQ ID NO:59, SEQ ID NO:73, SEQ ID NO:87, SEQ ID NO:103, SEQ ID NO:120, SEQ ID NO:137, or SEQ ID NO:154.

The present bispecific CAR may be encoded by a nucleic acid having a nucleotide sequence set forth in SEQ ID NO:45, SEQ ID NO:59, SEQ ID NO:73, SEQ ID NO:87, SEQ ID NO:103, SEQ ID NO:120, SEQ ID NO:137, or SEQ ID NO:154.

The present disclosure provides for an immune cell expressing or comprising the present bispecific CAR. The immune cell may be a T cell or a natural killer (NK) cell.

The present disclosure provides an immune cell, comprising the vector or the nucleic acid encoding the present CAR (e.g., integrated into its genome). The cell may be an isolated cell. The cell may be a genetically engineered cell. The cell may be a mammalian cell. In one embodiment, the cell is a CAR-T cell and/or a CAR-NK cell.

Also encompassed by the present disclosure is a nucleic acid encoding the present chimeric antigen receptor (e.g., the present bispecific CAR).

The present nucleic acid may comprise a nucleotide sequence about 80% to about 100%, about 85% to about 100%, about 90% to about 100%, or about 95% to about 100%, identical to the nucleotide sequence set forth in SEQ ID NO:45, SEQ ID NO:59, SEQ ID NO:73, SEQ ID NO:87, SEQ ID NO:103, SEQ ID NO:120, SEQ ID NO:137, or SEQ ID NO:154.

The present nucleic acid may comprise a nucleotide sequence set forth in SEQ ID NO:45, SEQ ID NO:59, SEQ ID NO:73, SEQ ID NO:87, SEQ ID NO:103, SEQ ID NO:120, SEQ ID NO:137, or SEQ ID NO:154.

The present disclosure provides for a vector comprising the present nucleic acid. The vector may comprise DNA or RNA. The vector may be a plasmid, virus vector, transposon, or combinations thereof. The vector may comprise a DNA virus or a retroviral vector. The vector may be a lentiviral vector, an adenoviral vector, an adeno-associated viral vector, or combinations thereof. In one embodiment, the vector is a lentiviral vector.

The present disclosure also provides for a pharmaceutical composition, comprising the present chimeric antigen receptor (e.g., the present bispecific CAR), the present immune cell, the present nucleic acid, or the present vector. The pharmaceutical composition may further comprise a pharmaceutically acceptable carrier, diluent or excipient. The pharmaceutical composition may be a liquid preparation.

The pharmaceutical composition may comprise the present immune cells at a concentration ranging from about 1×10³ cells/mL to about 1×10⁸ cells/mL, or from about 1×10⁴ cells/mL to about 1×10⁷ cells/mL.

The present disclosure also provides for a method of treating an autoimmune disease/disorder. The present disclosure provides for a method of treating cancer. The method may comprise administering the present immune cell or present pharmaceutical composition to a subject in need thereof.

The immune cell may be allogeneic or autologous.

The autoimmune disorder may be systemic lupus erythematosus (SLE) (e.g., lupus nephritis), systemic sclerosis (SSc), inflammatory myopathy (e.g., polymyositis, dermatomyositis, or inclusion-body myositis), systemic scleroderma, multiple sclerosis, or neuromyelitis optica (NMO).

The cancer may be a hematologic cancer. The cancer may be a B-cell malignancy. The cancer may be Hodgkin's lymphoma, non-Hodgkin's lymphoma, leukemia, and/or multiple myeloma. The cancer may be acute myeloid leukemia (AML), multiple myeloma (MM), chronic lymphocytic leukemia (CLL), chronic myelogenous leukemia, acute lymphoblastic leukemia (ALL), diffuse large B cell lymphoma (DLBCL), or combinations thereof.

The present disclosure provides a method for preparing an immune cell (e.g., a CAR-T cell) expressing the chimeric antigen receptor, where the method comprises: transducing the present nucleic acid molecule or the present vector into an immune cell (e.g., a T cell or NK cell), thereby obtaining the immune cell expressing the chimeric antigen receptor (e.g., the CAR-T cell).

The present disclosure provides a chimeric antigen receptor (CAR), wherein the structure of the chimeric antigen receptor may be shown in formula I:

$$SP - scFv 1 - linker 2 - scFv 2 - H - TM - C - CSD , \qquad ( I )$$

where, each “-” is independently a linker peptide or a peptide bond; SP is an optional signal peptide; H is an optional hinge region; TM is a transmembrane domain; C is a co-stimulatory region; CSD is a cytoplasmic signaling domain; one of scFv1 and scFv2 is an anti-BCMA antigen binding region, and the other is an anti-CD19 antigen binding region.

In one embodiment, scFv1 is an anti-BCMA antigen binding region, and scFv2 is an anti-CD19 antigen binding region. In another embodiment, scFv1 is an anti-CD19 antigen binding region, and scFv2 is an anti-BCMA antigen binding region.

Linker-2 may have the sequence set forth in SEQ ID NO: 30.

The structure of the anti-BCMA antigen binding region may be as shown in formula A or B as below:

$${V}_{H {1}^{-}} {V}_{L 1} ( A ) ; {V}_{L {1}^{-}} {V}_{H 1} ( B )$$

wherein V_{H1} is an anti-BCMA antibody heavy chain variable region; V_{L1} is an anti-BCMA antibody light chain variable region; and “-” is a linker peptide or a peptide bond.

In one embodiment, the present CAR has an anti-BCMA antigen binding region (or domain) with a structure as shown in formula B.

In certain embodiments, the amino acid sequence of V_{L1} is shown in SEQ ID NO: 24, and the amino acid sequence of V_{H1} is shown in SEQ ID NO: 28.

V_{L1} and V_{H1} may be linked with a linker peptide (linker 1 or linker-1). Linker-1 may have the sequence set forth in SEQ ID NO: 26.

The structure of the anti-CD19 antigen binding region may be as shown in formula C or D as below:

$${V}_{L {2}^{-}} {V}_{H 2} ( C ) ; {V}_{H {2}^{-}} {V}_{L 2} ( D )$$

where V_{L2} is an anti-CD19 antibody light chain variable region; V_{H2} is an anti-CD19 antibody heavy chain variable region; and “-” is a linker peptide or a peptide bond.

In one embodiment, the present CAR has an anti-CD19 antigen binding domain with a structure as shown in formula C.

In certain embodiments, the amino acid sequence of the V_{L2} is shown in SEQ ID NO: 32, and the amino acid sequence of the V_{H2} is shown in SEQ ID NO: 36.

V_{L2} and V_{H2} may be linked with a linker peptide (linker 3 or linker-3). Linker-3 may have the sequence set forth in SEQ ID NO: 34.

In another embodiment, the structure of the chimeric antigen receptor is shown in formula II as below:

$${SP}_{{}^{-}} {V}_{L {1}^{-}} {V}_{H {1}^{-}} linker {2}_{{}^{-}} {V}_{L {2}^{-}} {V}_{H {2}^{-}} {H}_{{}^{-}} {TM}_{{}^{-}} {C}_{{}^{-}} CSD \qquad ( II )$$

In one embodiment, linker 2 (or linker-2) has the sequence set forth in SEQ ID NO: 30.

In certain embodiments, the anti-BCMA antigen-binding region includes a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 24.

In certain embodiments, the anti-BCMA antigen-binding region includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 28.

In certain embodiments, the anti-CD19 antigen-binding region includes a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 32.

In certain embodiments, the anti-CD19 antigen-binding region includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO:36.

A light chain variable region (V_{L}) of the anti-BCMA antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in position 24-34, position 50-56, position 89-97 of SEQ ID NO:24, respectively (the CDRs of a light chain variable region of the BCMA-20 antibody).

A light chain variable region (V_{L}) of the anti-BCMA antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in SEQ ID NO: 2, SEQ ID NO: 4, SEQ ID NO: 6, respectively (the CDRs of a light chain variable region of the BCMA-20 antibody).

A heavy chain variable region (V_{H}) of the anti-BCMA antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in position 31-35, position 50-66, position 99-110 of SEQ ID NO:28, respectively (the CDRs of a heavy chain variable region of the BCMA-20 antibody).

A heavy chain variable region (V_{H}) of the anti-BCMA antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in SEQ ID NO: 9, SEQ ID NO: 11, SEQ ID NO: 13, respectively (the CDRs of a heavy chain variable region of the BCMA-20 antibody).

In certain embodiments, a light chain variable region (V_{L}) of the anti-BCMA antigen-binding region includes three CDRs, CDR1, CDR2 and CDR3, that are identical to CDR1, CDR2 and CDR3 as set forth in position 24-34, position 50-56, position 89-97 of SEQ ID NO: 24, respectively (CDRs of a light chain variable region of the BCMA-20 antibody), and a heavy chain variable region (V_{H}) of the anti-BCMA antigen-binding region includes three CDRs that are identical to CDR1, CDR2 and CDR3 as set forth in position 31-35, position 50-66, position 99-110 of SEQ ID NO: 28 (CDRs of a heavy chain variable region of the BCMA-20 antibody).

In certain embodiments, a light chain variable region (V_{L}) of the anti-BCMA antigen-binding region includes three CDRs, CDR1, CDR2 and CDR3, that are identical to CDR1, CDR2 and CDR3 as set forth in SEQ ID NO: 2, SEQ ID NO: 4, SEQ ID NO: 6, respectively (the CDRs of a light chain variable region of the BCMA-20 antibody), and a heavy chain variable region (V_{H}) of the anti-BCMA antigen-binding region includes three CDRs that are identical to CDR1, CDR2 and CDR3 as set forth in SEQ ID NO: 9, SEQ ID NO: 11, SEQ ID NO: 13, respectively (the CDRs of a heavy chain variable region of the BCMA-20 antibody).

A light chain variable region (V_{L}) of the anti-CD19 antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth position 24-34, position 50-56, position 89-97 of SEQ ID NO: 32, respectively (the CDRs of a light chain variable region of the FMC63 antibody).

A light chain variable region (V_{L}) of the anti-CD19 antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth SEQ ID NO: 15, SEQ ID NO: 16, SEQ ID NO: 17, respectively (the CDRs of a light chain variable region of the FMC63 antibody).

A heavy chain variable region (V_{H}) of the anti-CD19 antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in position 31-35, position 50-65, position 98-109 of SEQ ID NO: 36, respectively (the CDRs of a heavy chain variable region of the FMC63 antibody).

A heavy chain variable region (V_{H}) of the anti-CD19 antigen-binding region can comprise one, two, or three complementarity determining regions (CDRs), CDR1, CDR2 and CDR3, that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to CDR1, CDR2 and CDR3 as set forth in SEQ ID NO: 18, SEQ ID NO: 19, SEQ ID NO: 20, respectively (the CDRs of a heavy chain variable region of the FMC63 antibody).

In certain embodiments, a light chain variable region (V_{L}) of the anti-CD19 antigen-binding region includes three CDRs, CDR1, CDR2 and CDR3, that are identical to CDR1, CDR2 and CDR3 as set forth position 24-34, position 50-56, position 89-97 of SEQ ID NO: 32, respectively (CDRs of a light chain variable region of the FMC63 antibody), and a heavy chain variable region (V_{H}) of the anti-BCMA antigen-binding region includes three CDRs, CDR1, CDR2 and CDR3, that are identical to CDR1, CDR2 and CDR3 as set forth in position 31-35, position 50-65, position 98-109 of SEQ ID NO: 36, respectively (CDRs of a heavy chain variable region of the FMC63 antibody).

In certain embodiments, a light chain variable region of the anti-CD19 antigen-binding region includes three CDRs, CDR1, CDR2 and CDR3, that are identical to CDR1, CDR2 and CDR3 as set forth SEQ ID NO: 15, SEQ ID NO: 16, SEQ ID NO: 17, respectively (CDRs of a light chain variable region (V_{L}) of the FMC63 antibody), and a heavy chain variable region (V_{H}) of the anti-BCMA antigen-binding region includes three CDRs, CDR1, CDR2 and CDR3, that are identical to CDR1, CDR2 and CDR3 as set forth in SEQ ID NO: 18, SEQ ID NO: 19, SEQ ID NO: 20, respectively (CDRs of a heavy chain variable region of the FMC63 antibody).

In certain embodiments, the antigen-binding domain targeting BCMA in the present CAR comprises a light chain variable domain V_{L} (SEQ ID NO: 24) and a heavy chain variable domain V_{H} (SEQ ID NO: 28) derived from the BCMA-20 (B20) antibody.

The light chain variable domain V_{L} derived from the BCMA-20 antibody may have the below sequence:

(SEQ ID NO: 24)
DIQMTQSPSSLSASVGDRVTITCRASQGISNYLNWYQQKPGKAPKPLIY
YTSNLQSGVPSRFSGSGSGTDYTLTISSLQPEDFATYYCMGOTISSYTF
GQGTKLEIK

B20-VL-CDR1: SEQ ID NO: 24, position 24-34. The sequence of B20-VL-CDR1 is:

(SEQ ID NO: 2)
RASQGISNYLN.

B20-VL-CDR2: SEQ ID NO: 24, position 50-56. The sequence of B20-VL-CDR2 is:

(SEQ ID NO: 4)
YTSNLQS.

B20-VL-CDR3: SEQ ID NO: 24, position 89-97. The sequence of B20-VL-CDR3 is:

(SEQ ID NO: 6)
MGQTISSYT.

The heavy chain variable domain V_{H} derived from the BCMA-20 antibody may have the below sequence:

(SEQ ID NO: 28)
EVQLVESGGGLVQPGGSLRLSCAASGFTFSNFDMAWVRQAPGKGLVWVS
SITTGADHAIYADSVKGRFTISRDNAKNTLYLQMNSLRAEDTAVYYCVR
HGYYDGYHLFDYWGQGTLVTVSS

B20-VH-CDR1: SEQ ID NO: 28, position 31-35. The sequence of B20-VH-CDR1 is:

(SEQ ID NO: 9)
NFDMA.

B20-VH-CDR2: SEQ ID NO: 28, position 50-66. The sequence of B20-VH-CDR2 is:

(SEQ ID NO: 11)
SITTGADHAIYADSVKG.

B20-VH-CDR3: SEQ ID NO: 28, position 99-110. The sequence of B20-VH-CDR3 is:

(SEQ ID NO: 13)
HGYYDGYHLFDY.

In certain embodiments, the antigen-binding domain targeting CD19 in the present CAR comprises a light chain variable domain V_{L} (SEQ ID NO: 32) and a heavy chain variable domain V_{H} (SEQ ID NO: 36) derived from the FMC63 antibody.

The light chain variable domain V_{L} derived from the FMC63 antibody may have the below sequence:

(SEQ ID NO: 32)
DIQMTQTTSSLSASLGDRVTISCRASQDISKYLNWYQQKPDGTVKLL
IYHTSRLHSGVPSRFSGSGSGTDYSLTISNLEQEDIATYFCQQGNTL
PYTFGGGTKLEIT

FMC63-VL-CDR1: SEQ ID NO: 32, position 24-34. The sequence of FMC63-VL-CDR1 is: RASQDISKYLN (SEQ ID NO: 15).

FMC63-VL-CDR2: SEQ ID NO: 32, position 50-56. The sequence of FMC63-VL-CDR2 is:

(SEQ ID NO: 16)
HTSRLHS.

FMC63-VL-CDR3: SEQ ID NO: 32, position 89-97. The sequence of FMC63-VL-CDR3 is:

(SEQ ID NO: 17)
QQGNTLPYT.

The heavy chain variable domain VH derived from the FMC63 antibody may have the below sequence:

(SEQ ID NO: 36)
EVKLQESGPGLVAPSQSLSVTCTVSGVSLPDYGVSWIRQPPRKGLE
WLGVIWGSETTYYNSALKSRLTIIKDNSKSQVFLKMNSLQTDDTAI
YYCAKHYYYGGSYAMDYWGQGTSVTVSS

FMC63-VH-CDR1: SEQ ID NO: 36, position 31-35. The sequence of FMC63-VH-CDR1 is: DYGVS (SEQ ID NO: 18).

FMC63-VH-CDR2: SEQ ID NO: 36, position 50-65. The sequence of FMC63-VH-CDR2 is: VIWGSETTYYNSALKS (SEQ ID NO: 19).

FMC63-VH-CDR3: SEQ ID NO: 36, position 98-109. The sequence of FMC63-VH-CDR3 is: HYYYGGSYAMDY (SEQ ID NO: 20).

The signal peptide may be the signal peptide of CD8, having the following sequence:

(SEQ ID NO: 22)
MALPVTALLLPLALLLHAARP

The linker between V_{L} and V_{H} (or V_{H} and V_{L}) of the anti-BCMA scFv (linker-1) may have the following sequence: GGGGSGGGGSGGGGS (SEQ ID NO:26)

The linker between the anti-BCMA scFv and the anti-CD19 scFv (linker-2) may have the following sequence: GGGGS (SEQ ID NO:30)

The linker between V_{L} and V_{H} (or V_{H} and V_{L}) of the anti-CD19 scFv (linker-3) may have the following sequence: GGGGSGGGGSGGGGS (SEQ ID NO:34)

The hinge region between the extracellular region (antigen-binding domain) and the transmembrane domain may be derived from IgG4, CD8 (CD8a), CD28, CD137, or combinations thereof.

The hinge region may be derived from IgG4 which has the following sequence:

(SEQ ID NO: 38)
ESKYGPPCPPCP

The hinge region may be derived from CD8a which has the following sequence:

(SEQ ID NO: 98)
FVPVFLPAKPTTTPAPRPPTPAPTIASQPLSLRPEACRPAAGGAVH
TRGLDFACD

The transmembrane domain may be derived from CD28 (CD28TM) which has the following sequence: MFWVLVVVGGVLACYSLLVTVAFIIFWV (SEQ ID NO:40)

The transmembrane domain may be derived from CD8 (CD8TM) which has the following sequence: IYIWAPLAGTCGVLLLSLVITLYC (SEQ ID NO:100) The co-stimulatory region may be derived from 4-1BB which has the following sequence:

(SEQ ID NO: 42)
KRGRKKLLYIFKQPFMRPVQTTQEEDGCSCRFPEEEEGGCEL

The cytoplasmic signaling domain may be derived from CD3ζ (which has the following sequence:

(SEQ ID NO: 44)
RVKFSRSADAPAYQQGQNQLYNELNLGRREEYDVLDKRRGRDPEMG
GKPRRKNPQEGLYNELQKDKMAEAYSEIGMKGERRRGKGHDGLYQG
LSTATKDTYDALHMQALPPR

### Chimeric Antigen Receptors (CARs)

The terms “chimeric antigen receptor,” or alternatively “CAR”, are used interchangeably throughout and refer to a recombinant polypeptide construct comprising, e.g., an extracellular antigen binding domain, a transmembrane domain and an intracellular domain. Lee et al., Clin. Cancer Res. (2012) 18(10):2780; Jensen et al., Immunol Rev. (2014) 257(1):127. In one embodiment, the stimulatory molecule is the zeta chain associated with the T cell receptor complex. In one aspect, the cytoplasmic signaling domain further comprises one or more functional signaling domains derived from at least one costimulatory molecule. The costimulatory molecule may also be 4-1BB (i.e., CD137), CD27 and/or CD28 or fragments of those molecules. In another aspect, the CAR comprises a chimeric fusion protein comprising an extracellular antigen recognition domain, a transmembrane domain and an intracellular signaling domain comprising a functional signaling domain derived from a stimulatory molecule. The CAR may comprise a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. Alternatively, the CAR may comprise a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. The CAR may also comprise a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. The antigen-binding region of the CAR may contain any antigen-binding antibody fragment. The antibody fragment can comprise one or more CDRs, the variable region (or portions thereof), the constant region (or portions thereof), or combinations of any of the foregoing.

The term “zeta” or alternatively “zeta chain”, “CD3-zeta” or “TCR-zeta” may be the protein provided as GenBank accession numbers NP_932170, NP_000725, or XP_011508447; or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” may be the amino acid residues from the cytoplasmic domain of the zeta chain that are sufficient to functionally transmit an initial signal necessary for T cell activation.

A chimeric receptor may refer to a non-naturally occurring molecule that can be expressed on the surface of a host cell and comprises an antigen-binding fragment that binds to an antigen. In addition to the antigen-binding fragment, the chimeric receptor may further comprise one or more of a hinge region, a transmembrane domain, at least one co-stimulatory region, and a cytoplasmic signaling domain. In some embodiments, the chimeric antigen receptor comprises from N terminus to C terminus, an antigen-binding region (or fragment), a hinge region, a transmembrane domain, and a cytoplasmic signaling domain. In some embodiments, the chimeric antigen receptor further comprises at least one co-stimulatory region. Thus, the chimeric antigen receptor may comprise from N terminus to C terminus, an antigen-binding region (or fragment), a hinge region, a transmembrane domain, a co-stimulatory region, and a cytoplasmic signaling domain.

In some embodiments, the chimeric antigen receptors comprise a hinge region, which may be located between the antigen-binding region and a transmembrane domain. The hinge region may contain about 10-200 amino acids, e.g., 15-150 amino acids, 20-100 amino acids, or 30-60 amino acids. In some embodiments, the hinge region may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length. The hinge region may contain 0-300 amino acids, 2 amino acids to 100 amino acids, 5 amino acids to 80 amino acids, 10 amino acids to 60 amino acids, 10 amino acids to 15 amino acids, 20 amino acids to 80 amino acids, 30 amino acids to 70 amino acids, 40 amino acids to 60 amino acids, 50 amino acids to 60 amino acids, or 30 amino acids to 60 amino acids.

In some embodiments, the hinge region is a hinge domain of a naturally occurring protein. Hinge domains of any protein known in the art to comprise a hinge domain are compatible for use in the chimeric antigen receptors. In some embodiments, the hinge domain is of CD8α or CD28α. In some embodiments, the hinge domain is a portion of the hinge domain of CD8α, e.g., a fragment containing at least 15 (e.g., 20, 25, 30, 35, or 40) consecutive amino acids of the hinge domain of CD8α or CD28α.

Hinge domains of antibodies, such as an IgG, IgA, IgM, IgE, or IgD antibody, are also compatible for use in the chimeric antigen receptors. In some embodiments, the hinge region is the hinge domain that joins the constant domains CH1 and CH2 of an antibody. In some embodiments, the hinge region is of an antibody and comprises the hinge domain of the antibody and one or more constant regions of the antibody. In some embodiments, the hinge region comprises the hinge domain of an antibody and the CH3 constant region of the antibody. In some embodiments, the hinge region comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG4 antibody. In some embodiments, the hinge region comprises the hinge region and the CH3 constant region of an IgG4 antibody.

The hinge region may be a non-naturally occurring peptide. In some embodiments, the hinge region between the extracellular antigen-binding domain and the transmembrane domain is a peptide linker, such as a (GlyxSer)n (or (GxS)n) linker, wherein x and n, independently can be an integer between 3 and 12, including 3, 4, 5, 6, 7, 8, 9, 10, 11, 12, or more.

Additional peptide linkers that may be used in a hinge region of the chimeric receptors described herein are known in the art. See, e.g., Wriggers et al. Current Trends in Peptide Science (2005) 80(6): 736-746 and PCT Publication WO 2012/088461.

In some embodiments, the chimeric antigen receptors may comprise a transmembrane domain. The transmembrane domain can be in any form known in the art. Transmembrane domains compatible for use in the chimeric antigen receptors may be obtained from a naturally occurring protein. Alternatively, the transmembrane domain may be a synthetic, non-naturally occurring protein segment, e.g., a hydrophobic protein segment that is thermodynamically stable in a cell membrane.

In some embodiments, the transmembrane domain is that of CD8α. In some embodiments, the transmembrane domain is that of CD28. In some embodiments, the transmembrane domain is that of ICOS.

In some embodiments, the chimeric antigen receptors comprise one or more costimulatory regions. A co-stimulatory region may be at least a portion of a protein that mediates signal transduction within a cell to induce an immune response, such as an effector function. The co-stimulatory region of the chimeric antigen receptor can be from a protein which transduces a signal and modulates responses mediated by immune cells, such as T cells, natural killer (NK) cells, macrophages, neutrophils, or eosinophils.

In some embodiments, the chimeric antigen receptor comprises one or more than one (at least 2, 3, 4, or more) co-stimulatory region. In some embodiments, the chimeric antigen receptor comprises more than one co-stimulatory region obtained from different proteins. In some embodiments, the chimeric antigen receptor does not comprise a co-stimulatory region.

Examples of co-stimulatory regions for use in the chimeric antigen receptors can be a domain from co-stimulatory proteins, including, without limitation, CD27, CD28, 4-1BB, OX40, CD30, Cd40, PD-1, ICOS, lymphocyte function-associated antigen-1 (LFA-1), CD2, CD7, LIGHT, NKG2C, B7-H3. In some embodiments, the co-stimulatory region is derived from 4-1BB, CD28, or ICOS. In some embodiments, the co-stimulatory region is derived from CD28 and the chimeric antigen receptor comprises a second co-stimulatory region from 4-1BB or ICOS. In some embodiments, the co-stimulatory region is a fusion domain comprising more than one co-stimulatory region or portions of more than one co-stimulatory region. In some embodiments, the costimulatory region is a fusion of costimulatory regions from CD28 and ICOS.

In some embodiments, the chimeric antigen receptors comprise a cytoplasmic signaling domain. Any cytoplasmic signaling domain can be used in the chimeric antigen receptors described herein. A cytoplasmic signaling domain may relay a signal, such as interaction of an extracellular ligand-binding domain with its ligand, to stimulate a cellular response, such as inducing an effector function of the cell (e.g., cytotoxicity).

The chimeric antigen receptors can be prepared by routine methods, such as recombinant technology. Methods for preparing the chimeric antigen receptors may involve generation of a nucleic acid that encodes a polypeptide comprising each of the domains of the chimeric antigen receptors, including the antigen-binding fragment and optionally, the hinge region, the transmembrane domain, at least one co-stimulatory region, and the cytoplasmic signaling domain. In some embodiments, nucleic acids encoding each of the components of the chimeric antigen receptor are joined together using recombinant technology. Sequences of each of the components (e.g., domains) can be joined directly or indirectly (e.g., using a nucleic acid sequence encoding a peptide linker) to form a nucleic acid sequence encoding the chimeric antigen receptor, using methods such as PCR amplification or ligation. Alternatively, the nucleic acid encoding the chimeric antigen receptor may be synthesized. In some embodiments, the nucleic acid is DNA. In other embodiments, the nucleic acid is RNA.

In one embodiment, the present CAR, from the N-terminus to C-terminus, comprises a signal peptide (also known as leader sequence), an antigen recognition sequence (antigen-binding domain), a hinge region, a transmembrane domain, a co-stimulatory region, and a cytoplasmic signaling domain (e.g., a CD3zeta signaling region ((chain portion)).

Bispecificity means that the CAR can specifically bind two different antigens. The bispecific CAR may generate an immune response by binding to one antigen or both antigens.

As used herein, the terms “CAR-T cell”, “CAR-T”, “CART”, “CART cell” may refer to the T cell that expresses the present CAR targeting both BCMA and CD19.

### Immune Cells Expressing Chimeric Antigen Receptors

The present disclosure also provides immune cells expressing the present CAR. Recognition of a target cell having the antigen(s) on its cell surface by the antigen-binding fragment of the chimeric antigen receptor may transduce an activation signal to the signaling domain(s) (e.g., co-stimulatory region and/or the cytoplasmic signaling domain) of the chimeric antigen receptor, which may activate an effector function in the immune cell expressing the chimeric antigen receptor.

The chimeric antigen receptor can be introduced into a suitable immune cell for expression via conventional technology. In some embodiments, the immune cells are T cells, such as primary T cells or T cell lines. Alternatively, the immune cells can be natural killer (NK) cells, such as established NK cell lines (e.g., NK-92 cells). In some embodiments, the immune cells are T cells that express CD8 (CD8⁺) or CD8 and CD4 (CD8⁺/CD4⁺). In some embodiments, the T cells are T cells of an established T cell line, for example, Jurkat cells.

Primary T cells may be obtained from any source, such as peripheral blood mononuclear cells (PBMCs), bone marrow, tissues such as spleen, lymph node, thymus, or tumor tissue. In some embodiments, the population of immune cells is derived from a human patient having an autoimmune disorder or cancer (e.g., hematopoietic malignancy), such as from the bone marrow or from PBMCs obtained from the patient. In some embodiments, the population of immune cells is derived from a healthy donor. In some embodiments, the immune cells are obtained from the subject to whom the immune cells expressing the chimeric antigen receptors will be subsequently administered. Immune cells that are administered to the same subject from which the cells were obtained are referred to as autologous cells, whereas immune cells that are obtained from a subject who is not the subject to whom the cells will be administered may be referred to as allogeneic cells.

The type of immune cells desired may be expanded within the population of cells obtained by co-incubating the cells with stimulatory molecules, for example, anti-CD3 and anti-CD28 antibodies may be used for expansion of T cells.

To construct the immune cells that express the chimeric antigen receptors described herein, vectors for stable or transient expression of the chimeric antigen receptor may be constructed via conventional methods as described herein and introduced into immune cells. For example, nucleic acids encoding the chimeric antigen receptors may be cloned into a suitable vector, such as a viral vector.

In certain embodiments, immune cells (e.g., T cells) are transduced with lentiviral vectors (LVs) encoding the present CAR. The transduced immune cells (e.g., T cells) can target CD19 and BCMA, synergistically activate the T cells, and induce T cell-mediated immune responses.

In one embodiment, in the present method, T cells from an autologous patient (or an allogeneic donor) are isolated, activated and genetically modified to generate CAR-T cells expressing the present CAR, and then administered to the patient. CAR-T cells can replicate in vivo resulting in long-term persistence. In addition, the CAR-mediated immune response may be part of an adoptive immunotherapy approach in which the anti-BCMA/CD19 CAR-T cells elicit an immune response against cells expressing BCMA and/or CD19.

In certain embodiments, cells are isolated from a mammal (e.g., a human) and genetically modified (i.e., transduced or transfected in vitro) with a vector expressing a CAR disclosed herein. The CAR-modified cells can be administered to a mammalian recipient to provide a therapeutic benefit. The mammalian recipient may be a human. The CAR-modified cell can be autologous with respect to the recipient. Alternatively, the cells can be allogeneic, syngeneic or xenogeneic with respect to the recipient.

The methods of preparing immune cells expressing the present chimeric antigen receptors may comprise activating and/or expanding the immune cells ex vivo. Activating an immune cell means stimulating an immune cell into an activated state in which the cell may be able to perform effector functions (e.g., cytotoxicity). Methods of activating an immune cell will depend on the type of the immune cell used for expression of the chimeric antigen receptors. Expanding immune cells may involve any method that results in an increase in the number of cells expressing chimeric antigen receptors, for example, allowing the cells to proliferate or stimulating the cells to proliferate. In some embodiments, the cells expressing the chimeric receptors described herein are activated and/or expanded ex vivo prior to administration to a subject.

The CAR-expressing immune cells may also serve as a vaccine for ex vivo immunization and/or in vivo therapy in a mammal. In addition to using a cell-based vaccine in terms of ex vivo immunization, the present disclosure also provides compositions and methods for in vivo immunization to elicit an immune response directed against an antigen in a patient. Preferably, the mammal is a human. With respect to ex vivo immunization, one or more of the following may occur in vitro prior to administering the cell into a mammal: i) expanding the cells, ii) introducing a nucleic acid encoding a CAR to the cells, and/or iii) cryopreservation of the cells.

### Vectors

The present disclosure provides a nucleic acid encoding the present CAR. The present disclosure also provides vectors comprising the present nucleic acid.

The vectors include, but are not limited to, a plasmid, a phagemid, a phage derivative, a virus, and a cosmid.

The vector may be a viral vector. Viruses, which are useful as vectors comprise, but are not limited to, retroviruses, adenoviruses, adeno-associated viruses, herpes viruses, and lentiviruses. In certain embodiments, the present vector is a retroviral vector such as a lentiviral vector. In some embodiments, the vectors for expression of the chimeric antigen receptors are retroviruses. In some embodiments, the vectors for expression of the chimeric antigen receptors are lentiviruses. In some embodiments, the vectors for expression of the chimeric antigen receptors are adeno-associated viruses.

A variety of promoters can be used for expression of the chimeric receptors, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter (e.g., inducible promoters) may be used, such that its expression can be modulated within an immune cell.

The vector can be introduced into a cell, e.g., mammalian, bacterial, yeast, or insect cell, by any method in the art. For example, the vector can be transferred into a cell by physical, chemical, or biological means.

Physical methods for introducing a polynucleotide into a cell comprise calcium phosphate precipitation, lipofection, particle bombardment, microinjection, electroporation, and the like. See, for example, Sambrook et al. (2001, Molecular Cloning: A Laboratory Manual, Cold Spring Harbor Laboratory, New York).

Biological methods for introducing a polynucleotide of interest into a cell comprise the use of DNA and RNA vectors. Viral vectors can be derived from retroviruses, lentiviruses, poxviruses, herpes simplex virus I, adenoviruses and adeno-associated viruses, and the like.

Chemical means for introducing a polynucleotide into a host cell comprise colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, the vector (nucleic acid) encoding the chimeric antigen receptor is a DNA vector and may be electroporated to immune cells (see, e.g., Till, et al. Blood (2012) 119(17): 3940-3950). In some embodiments, the vector (nucleic acid) encoding the chimeric antigen receptor is an RNA molecule, which may be electroporated to immune cells.

Any of the vectors comprising a nucleic acid that encodes a chimeric antigen receptor described herein is also within the scope of the present disclosure. Such a vector may be delivered into host cells such as immune cells by a suitable method. Methods of delivering vectors to immune cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. PNAS (2013) 110(6): 2082-2087); or viral transduction. In some embodiments, the vectors for expression of the chimeric receptors are delivered to cells by viral transduction.

In examples in which the vectors encoding chimeric antigen receptors are introduced to the host cells using a viral vector, viral particles that are capable of infecting the immune cells and carry the vector may be produced by any method known in the art. The viral particles are harvested from the cell culture supernatant and may be isolated and/or purified prior to contacting the viral particles with the immune cells.

### Pharmaceutical Compositions

The present disclosure provides a pharmaceutical composition comprising the present immune cells, the present CAR, the present nucleic acid, or the present vector. The present pharmaceutical composition may further comprise a pharmaceutically acceptable carrier, diluent or excipient. In one embodiment, the preparation is a liquid preparation. In one embodiment, the concentration of the immune cells (e.g., CAR-T cells) in the preparation is 1×10³ to 1×10⁸ cells/mL, or 1×10⁴ to 1×10⁷ cells/mL.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of a disease or disorder in the subject. In some embodiments, the subject is a mammal. In some embodiments, the subject is a human.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

The present pharmaceutical composition may be delivered to a cell by contacting the cell with the present pharmaceutical composition.

The present pharmaceutical composition may be delivered/administered to a subject by any route, including, without limitation, intravenous, intracerebroventricular (ICV) injection, intracisternal injection or infusion, oral, transdermal, ocular, intraperitoneal, subcutaneous, implant, sublingual, subcutaneous, intramuscular, rectal, mucosal, ophthalmic, intrathecal, intra-articular, intra-arterial, sub-arachinoid, bronchial and lymphatic administration. The present pharmaceutical composition may be administered parenterally or systemically. The present composition may be administered locally. The pharmaceutical composition may be formulated for intravenous administration.

The administration of the present compositions may be carried out in any convenient manner, including by aerosol inhalation, injection, ingestion, transfusion, implantation or transplantation. The compositions may be administered to a patient subcutaneously, intradermally, intratumorally, intranodally, intramedullary, intramuscularly, by intravenous (i.v.) injection, or intraperitoneally. In one embodiment, the compositions are administered to a subject (e.g., a patient) by intradermal or subcutaneous injection. In another embodiment, the compositions are administered by i.v. injection. The compositions may be injected directly into a tumor, lymph node, or site of disorder.

The present immune cells or pharmaceutical composition may be delivered/administered to a subject via intravenous, intramuscular, subcutaneous, intraperitoneal, spinal or other parenteral administration, such as by injection or infusion.

The present pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an effective amount”, “a therapeutically effective amount”, or “a therapeutic amount” is indicated, the precise amount of the compositions to be administered can be determined by a physician with consideration of individual differences in age, weight, tumor size, extent of infection or metastasis, and condition of the patient (subject). A pharmaceutical composition comprising the immune cells may be administered at a dosage of 10⁴ to 10⁹ cells/kg body weight, or 10¹ to 10⁶ cells/kg body weight, including all integer values within those ranges. The compositions may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

The dosage of the above treatments to be administered to a patient may vary with the precise nature of the condition being treated and the recipient of the treatment. The scaling of dosages for patient administration can be performed according to art-accepted practices. In one embodiment, 1×10⁶ to 1×10¹⁰ of the immune cells (e.g., CAR-T cells) can be administered to a patient by means of, for example, intravenous infusion for each treatment or each course of treatment.

### Conditions to be Treated

The present CAR, immune cells or pharmaceutical composition may be used to treat an autoimmune disease/disorder, or to treat cancer or tumor.

In certain embodiments, the present anti-BCMA/CD19 bispecific CAR targets both B cells and plasma cells, which may reduce/eradicate autoimmune antibodies. In certain embodiments, the present anti-BCMA/CD19 bispecific CAR may reduce/deplete B cells, plasmablasts, and/or long-lived plasma cells (LLPCs) to reduce/eradiate autoantibody production.

The present disclosure provides for a method of treating an autoimmune disease/disorder. The method may comprise administering the CAR, immune cells or pharmaceutical composition to a subject in need thereof.

The autoimmune disorder may be systemic lupus erythematosus (SLE), lupus nephritis (LN), systemic sclerosis (SSc), CREST syndrome (calcinosis, Raynaud's syndrome, esophageal dysmotility, sclerodactyl, and telangiectasia), opsoclonus, inflammatory myopathy (e.g., polymyositis, dermatomyositis, and inclusion-body myositis), myositis autoantibody-driven diseases, systemic scleroderma, primary biliary cirrhosis, celiac disease (e.g., gluten sensitive enteropathy), dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis (MG), Lambert-Eaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, neuromyelitis optica (NMO), chronic inflammatory bowel disease, Hashimoto's disease, organ transplant rejection, and/or neuromyelitis optica spectrum disorder (NMOSD).

The autoimmune disorder may be pernicious anemia, Addison's disease, psoriasis, inflammatory bowel disease (IBD), psoriatic arthritis, Sjögren's syndrome, lupus erythematosus (e.g., discoid lupus erythematosus, drug-induced lupus erythematosus, and neonatal lupus erythematosus), multiple sclerosis, and/or reactive arthritis.

The autoimmune disorder may be polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, alopecia arcata, pemphigoid, scleroderma, progressive systemic sclerosis, adult onset diabetes mellitus (e.g., type II diabetes), male and female autoimmune infertility, ankylosing spondolytis, ulcerative colitis, Crohn's disease, sprue, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, antiphospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and/or autoimmune gonadal failure.

The autoimmune diseases also include, e.g., acute disseminated encephalomyelitis, alopecia areata, antiphospholipid syndrome, autoimmune hepatitis, autoimmune myocarditis, autoimmune pancreatitis, autoimmune polyendocrine syndromesautoimmune uveitis, inflammatory bowel disease (Crohn's disease, ulcerative colitis), type I diabetes mellitus (e.g; juvenile onset diabetes), multiple sclerosis, scleroderma, ankylosing spondylitis, sarcoid, pemphigus vulgaris, pemphigoid, psoriasis, myasthenia gravis, systemic lupus erythematosus, rheumatoid arthritis, juvenile arthritis, psoriatic arthritis, Behcet's syndrome, Reiter's disease, Berger's disease, dermatomyositis, polymyositis, antineutrophil cytoplasmic antibody-associated vasculitides (e.g., granulomatosis with polyangiitis (also known as Wegener's granulomatosis), microscopic polyangjitis, and Churg-Strauss syndrome), scleroderma, Sjogren's syndrome, anti-glomerular basement membrane disease (including Goodpasture's syndrome), dilated cardiomyopathy, primary biliary cirrhosis, thyroiditis (e.g., Hashimoto's thyroiditis, Graves' disease), transverse myelitis, allergies, arthritis, fibromyalgia, fibromytosis, lupus, vitiligo, and Guillane-Barre syndrome.

The autoimmune diseases include inflammatory bowel disease (IBD), ulcerative colitis, Crohn's disease, sprue, autoimmune arthritis, rheumatoid arthritis, Type I diabetes, multiple sclerosis, graft vs. host disease following bone marrow transplantation, osteoarthritis, juvenile chronic arthritis, Lyme arthritis, psoriatic arthritis, reactive arthritis, spondy loarthropathy, systemic lupus erythematosus, insulin dependent diabetes mellitus, thyroiditis, asthma, psoriasis, dermatitis scleroderma, atopic dermatitis, graft versus host disease, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, septic shock, toxic shock syndrome, sepsis syndrome, cachexia, acquired immunodeficiency syndrome, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrorme, adult (acute) respiratory distress syndrome, alopecia, alopecia areata, seronegative arthopathy, arthropathy, Reiter's disease, psoriatic arthropathy, chlamydia, yersinia and salmonella associated arthropathy spondyloarhopathy, atheromatous disease/arteriosclerosis, atopic allergy, food allergies, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondy litis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, multiple sclerosis (all subtypes), insulindependent diabetes mellitus, sympathetic ophthalmia, pulmonary hypertension secondary to connective tissue disease, Goodpasture's syndrome, pulmonary manifestation of polyarteritis nodosa, acute rheumatio fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, allergic rhinitis (pollen allergies), anaphylaxis, pet allergies, latex allergies, drug allergies, allergic rhinoconjuctivitis, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis.

The autoimmune disorder may be an inflammatory muscle disease. Inflammatory myopathies are a group of diseases that involve chronic muscle inflammation, muscle weakness, and in some cases, muscle pain. The four main types of chronic, or long-term, inflammatory myopathy are: polymyositis, which affects skeletal muscles (the type involved in body movement) on both sides of the body; dermatomyositis, which causes progressive muscle weakness; inclusion body myositis, which is characterized by slow, progressive muscle weakness and muscle shrinking and loss of muscle; and necrotizing autoimmune myopathy, which involves muscle weakness in the upper and lower body.

In another embodiment, the autoimmune disease is an autoimmune disease caused by overexpression of B cells (such as lupus erythematosus).

Also encompassed by the present disclosure is a method of treating cancer. The method may comprise administering the CAR, immune cells or pharmaceutical composition to a subject in need thereof.

The present disclosure provides chimeric antigen receptors for treating CD19-positive diseases such as B cell lymphoma.

The cancer may be a BCMA-positive malignancy. The cancer may be multiple myeloma (MM), or plasma cell leukemia.

The cancer may be a hematologic cancer. The cancer may be a plasma-cell malignancy. The cancer may be a B-cell malignancy. The B-cell malignancy may be acute lymphocytic leukemia (ALL), chronic lymphocytic leukemia (CLL), B-cell acute lymphoblastic leukemia (B-ALL), B-cell leukemia, or B cell lymphoma.

The cancer may be Hodgkin's lymphoma, non-Hodgkin's lymphoma, leukemia, and/or multiple myeloma (MM).

The cancer may be acute myeloid leukemia (AML), multiple myeloma (MM), chronic lymphocytic leukemia (CLL), chronic myelogenous leukemia, acute lymphoblastic leukemia (ALL), diffuse large B cell lymphoma (DLBCL), or a combination thereof.

Diseases that may be treated using the present CAR, immune cells or pharmaceutical composition include CD19-positive tumors and diseases, e.g., caused by excessive B cells (such as autoimmune diseases, for example, lupus erythematosus, etc.). CD19-positive tumors may include CD19-positive non-solid tumors (such as hematological cancer, for example, leukemias and lymphomas) or solid tumors. Tumors or cancers to be treated with present CAR, immune cells or pharmaceutical composition include, but are not limited to, carcinoma, blastoma, and sarcoma, and leukemia or lymphoid malignancies, benign and malignant tumors, and malignancies e.g., sarcomas, carcinomas, gastric cancer, peritoneal metastasis of gastric cancer, liver cancer, renal cancer, lung cancer, small intestine cancer, bone cancer, prostate cancer, colorectal cancer, breast cancer, large intestine cancer, cervical cancer, ovarian cancer, lymphoma, nasopharyngeal carcinoma, adrenal tumor, bladder tumor, non-small cell lung cancer (NSCLC), glioma, endometrial cancer, and melanomas. Adult tumors/cancers and pediatric tumors/cancers are included.

Hematologic cancers are cancers of the blood or bone marrow. Examples of hematological (or hematogenous) cancers include leukemias, e.g., acute leukemias (such as acute lymphocytic leukemia, acute myelocytic leukemia, acute myelogenous leukemia and myeloblasts, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

The cancer may be a solid tumor. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, mesothelioma, malignant lymphoma, pancreatic cancer and ovarian cancer.

### Kits

Also within the scope of the present disclosure are kits for use of the present CARs, immune cells, nucleic acids, vectors or pharmaceutical compositions. Such kits may include one or more containers comprising the present CARs, immune cells, nucleic acids, vectors or pharmaceutical compositions.

In some embodiments, the kit can comprise instructions for use in any of the methods described herein. The included instructions can comprise a description of administration of the pharmaceutical composition to a subject to achieve the intended activity in a subject. The kit may further comprise a description of selecting a subject suitable for treatment based on identifying whether the subject is in need of the treatment. In some embodiments, the instructions comprise a description of administering the pharmaceutical compositions to a subject who is in need of the treatment.

The instructions relating to the use of the pharmaceutical compositions generally include information as to dosage, dosing schedule, and route of administration for the intended treatment. The containers may be unit doses, bulk packages (e.g., multi-dose packages) or sub-unit doses.

The kits provided herein are in suitable packaging. Suitable packaging includes, but is not limited to, vials, bottles, jars, flexible packaging, and the like.

The following examples of specific aspects for carrying out the present disclosure are offered for illustrative purposes only, and are not intended to limit the scope of the present disclosure in any way.

### Example 1 Construction of Anti-BCMA/CD19 CARs

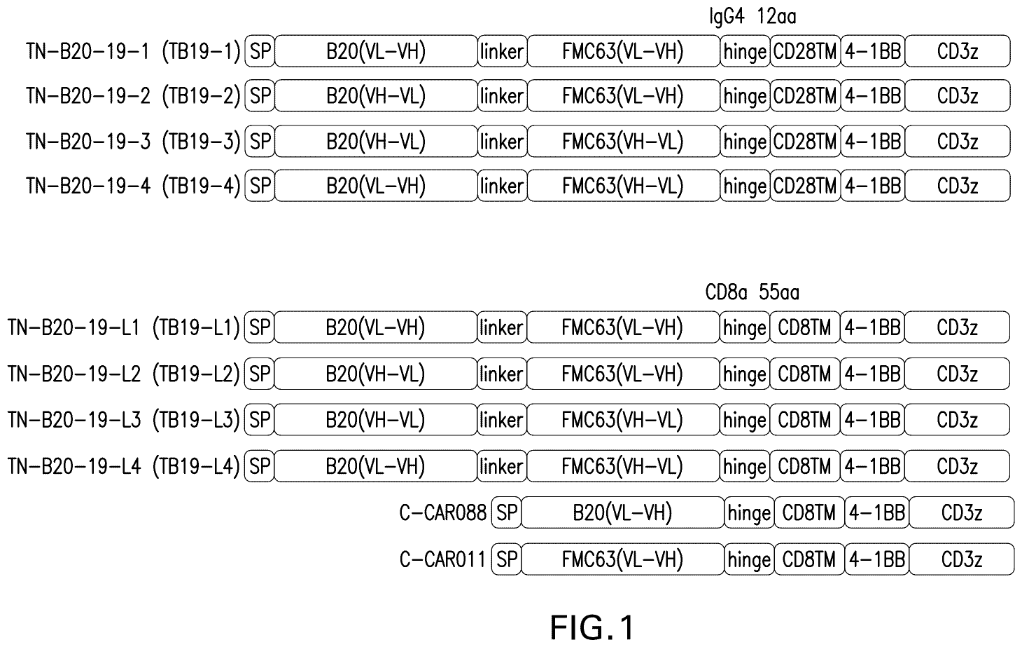

We prepared eight bispecific CARs having the anti-BCMA scFv and anti-CD19 scFv in the same order (i.e., anti-BCMA scFv (“B20”) followed by anti-CD19 scFv (“FMC63”)), but with different V_{H}/V_{L} orders and having different hinge regions and/or transmembrane domains: TB19-1 to TB19-4 and TB19-L1 to TB19-L4 (FIG. 1).

The anti-BCMA/CD19 CAR-T cells were prepared using apheresis from healthy donors.

Specifically, PBMCs were isolated from the venous blood of healthy donors by density gradient centrifugation. On day 0, PBMCs were activated in a cell culture flask previously coated with CD3 monoclonal antibody (OKT3) and Retronectin (TAKARA). The medium was GT-551 cell culture medium containing 1% human albumin and 300 U/mL recombinant human interleukin 2 (IL-2). On day 3, activated PBMCs were transduced with lentiviral vectors encoding the anti-BCMA/CD19 CARs.

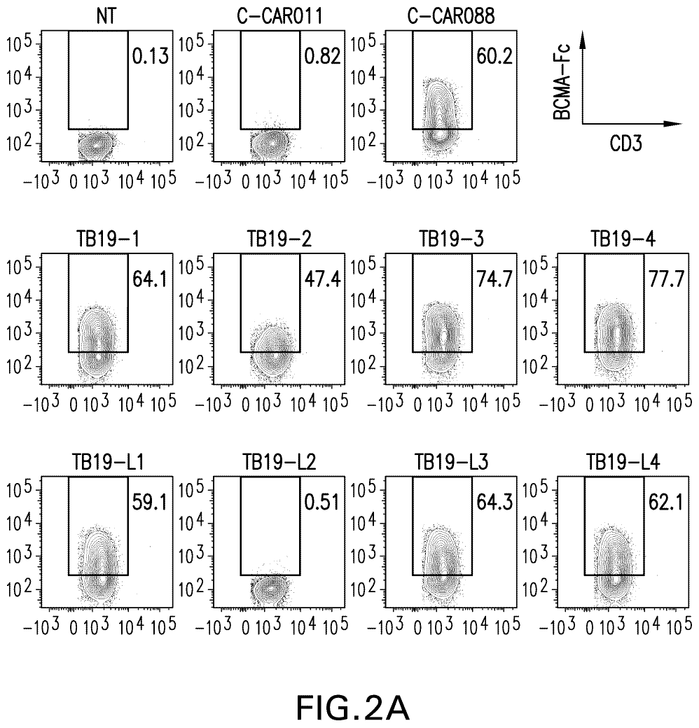

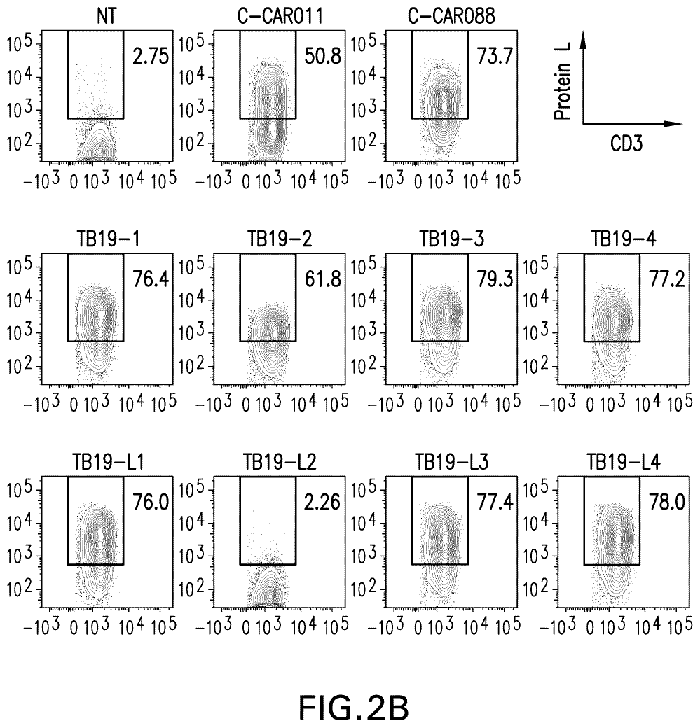

FIGS. 2A-2B show the expression levels of the anti-BCMA and anti-CD19 on the surface of T cells. The expression levels of anti-BCMA CARs were detected by flow cytometry using BCMA-Fc fusion protein; the expression levels of anti-CD19 CARs were detected by flow cytometry using Protein L.

### Example 2 Antigen-Specific Activation of Anti-BCMA/CD19 CAR-T Cells In Vitro

Antigen-specific activation of the anti-BCMA/CD19 CAR-T was evaluated by assaying IFN-γ release and CD137 expression when the CAR-T cells were co-cultured with target cells. Target cells (“T”) included CD19-positive A549-CD19 tumor cells, BCMA-positive A549-BCMA tumor cells, CD19 and BCMA double positive A549-CD19-BCMA tumor cells, Raji cells, MM.1S cells, and double negative A549 tumor cells. Effector cells (“E”) are the anti-BCMA/CD19 CAR-T cells.

PBMCs were isolated from the venous blood of healthy donors by density gradient centrifugation. On day 0, PBMCs were activated in a cell culture flask previously coated with CD3 monoclonal antibody (OKT3) and Retronectin (TAKARA). The medium was GT-551 cell culture medium containing 1% human albumin and 300 U/mL recombinant human interleukin 2 (IL-2). On day 3, activated PBMCs were transduced with lentiviral vectors encoding the anti-BCMA/CD19 CARs. Starting from day 6, the CAR-T cells can be taken for activity assays.

IFNγ release was assayed using the CAR-T cells cultured for 7 days. 1×10⁵ of CAR-T cells were cultured with CD19-positive A549-CD19 tumor cells, BCMA-positive A549-BCMA tumor cells, CD19 and BCMA double positive A549-CD19-BCMA tumor cells, double negative A549 tumor cells, or without tumor cells (NT), in 200 μl of medium for 18 h with an E:T ratio of 1:1. Then the levels of IFN-γ secreted in the cell culture supernatant were detected by ELISA.

Expression levels of CD137 were assayed using the CAR-T cells cultured for 7 days. 1×10⁵ of CAR-T cells were cultured with CD19-positive A549-CD19 tumor cells, BCMA-positive A549-BCMA tumor cells, CD19 and BCMA double positive A549-CD19-BCMA tumor cells, double negative A549 tumor cells, or without tumor cells, in 200 μl of medium for 18 h with an E:T ratio of 1:1. Then the expression levels of CD137 on the surface of the CAR-T cells were detected by flow cytometry.

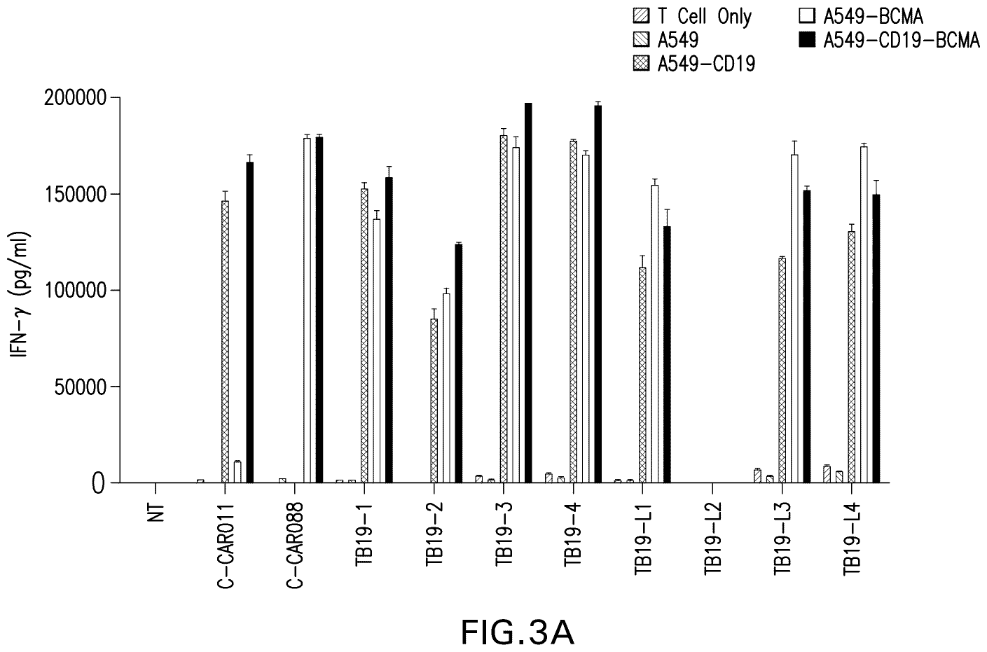

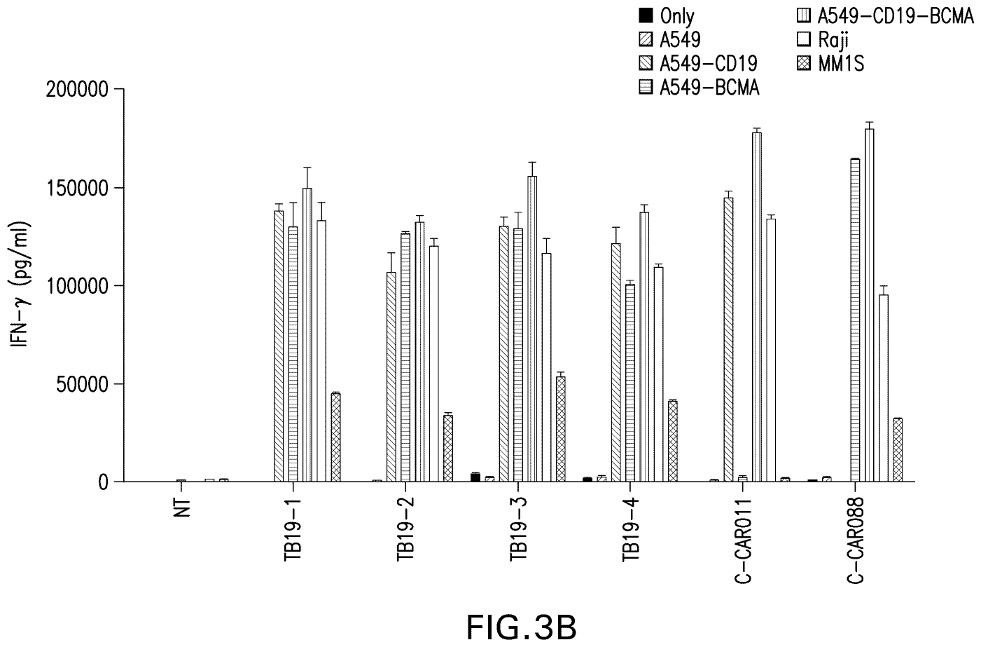

The IFNγ release results are shown in FIGS. 3A-3B. After co-culturing the CAR-T cells with A549 cells expressing CD19 and/or BCMA antigens, anti-CD19/BCMA CAR-T (TB19-1 to 4 and TB19-L1 to L4) cells can recognize CD19 single-positive, BCMA single-positive and CD19/BCMA double-positive target cells, as well as release high levels of IFN-γ. TB19-1 to TB19-4 showed high IFN-γ release when co-cultured with CD19/BCMA positive targets cells.

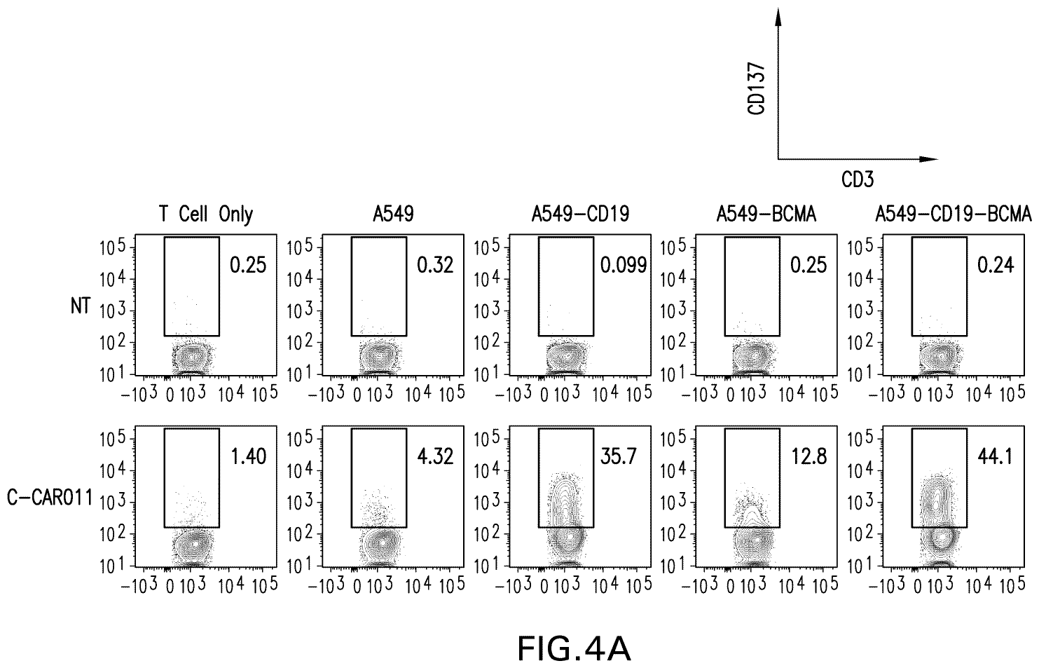

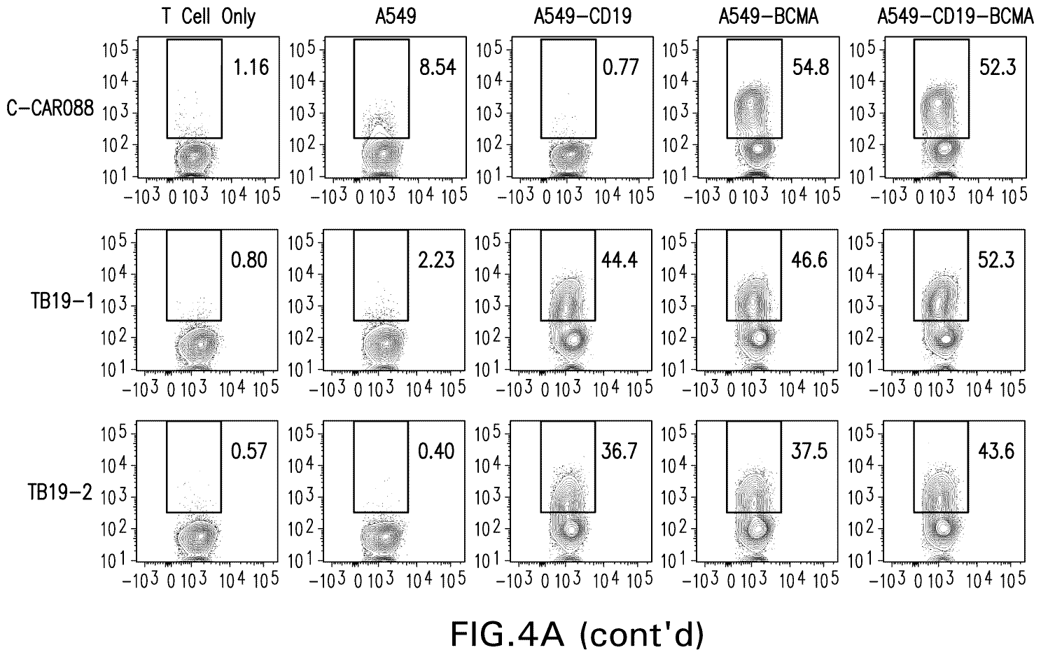

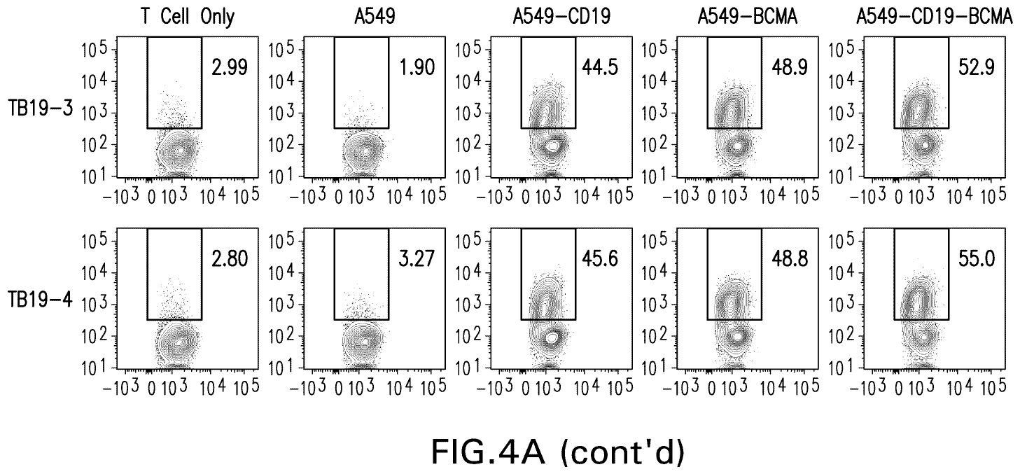

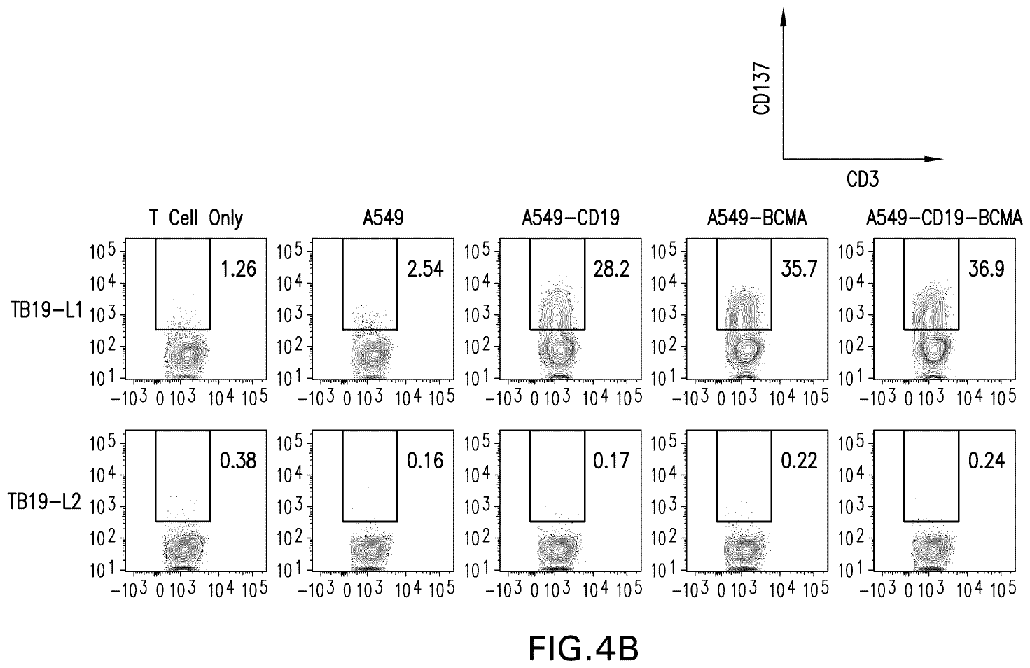

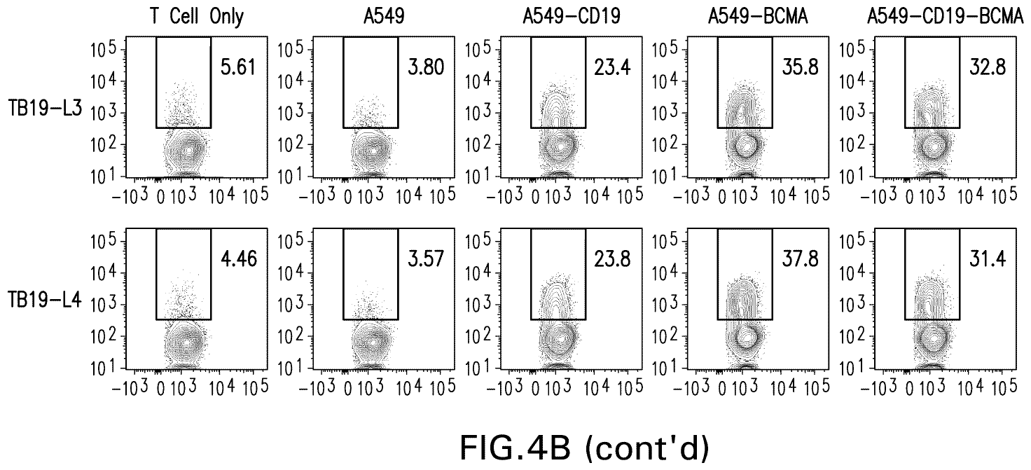

The flow cytometry results showed that the anti-CD19/BCMA CAR-T cells were activated by a variety of CD19/BCMA single-positive or double-positive cells and up-regulated the expression level of CD137 (FIGS. 4A-4B).

### Example 3 Cytotoxicity of Anti-CD19/BCMA CAR-T Cells In Vitro

The anti-BCMA/CD19 CAR-T cells were co-cultured with target cells at E:T ratios of 0:1, 1:1, 2:1 and 4:1, respectively. Real-Time Cell Analysis (RTCA) label-free technology was used to evaluate the cytotoxicity of the CAR-T cells on target cells.

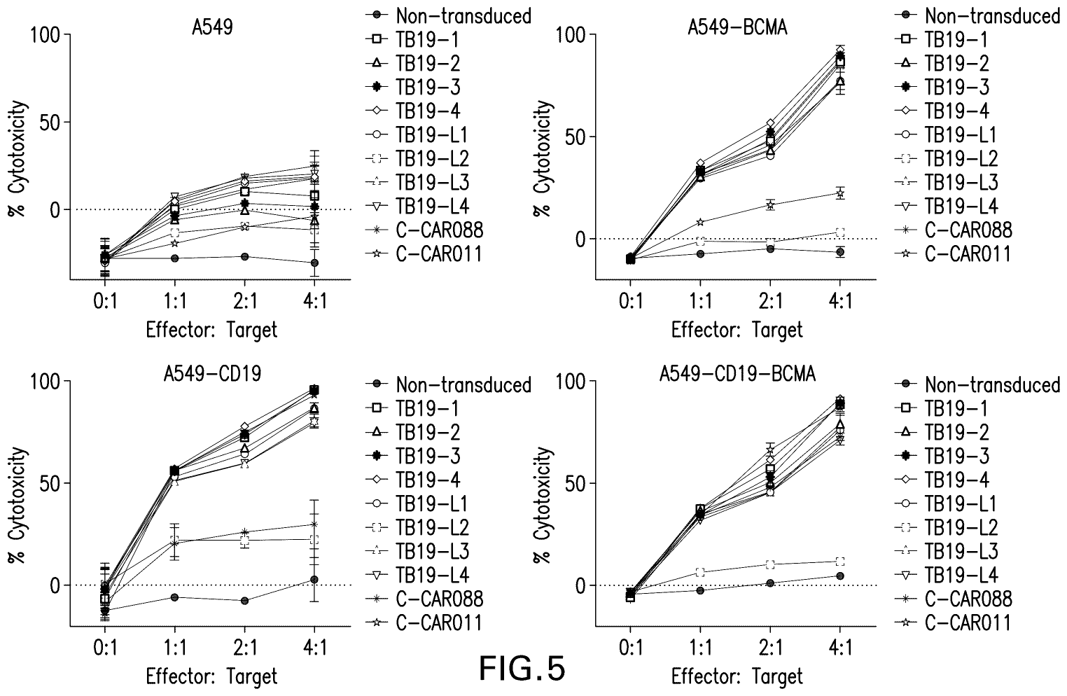

The results show that the anti-BCMA/CD19 CAR-T cells effectively killed BCMA/CD19 single-positive or double-positive tumor cells in vitro (A549-CD19, A549-BCMA, A549-BCMA-CD19), while they had no effect on A549 cells which do not express CD19 or BCMA (FIG. 5).

### Example 4 Cytotoxicity of Anti-BCMA/CD19 CAR-T Cells on Autoreactive B Cells In Vitro

Recent studies have shown that in patients with systemic lupus erythematosus (SLE), the proportion of CD11c^{hi}T-bet⁺ B cell subsets is significantly increased, and is closely related to the production of autoantibodies and the patient's clinical manifestations. Autoantibodies are characteristics of reactive B cells (see, Distinct Effector B Cells Induced by Unregulated Toll-like Receptor 7 Contribute to Pathogenic Responses in Systemic Lupus Erythematosus, Immunity, 2018, 16; 49(4):725-739.e6. IL-21 drives expansion and plasma cell differentiation of autoreactive CD11chiT-bet+ B cells in SLE, Nat. Commun. 2018; 9(1):1758). This subset of cells is enriched with age in some animal models of autoimmune diseases and in the peripheral blood of patients with rheumatoid arthritis, so they are also called age-associated B cells (ABCs) (see, Toll-like receptor 7 (TLR7)-driven accumulation of a novel CD11c⁺ B-cell population is important for the development of autoimmunity. Blood, 2011; 118(5):1305-15. A B-cell subset uniquely responsive to innate stimuli accumulates in aged mice, Blood, 2011; 118(5):1294-304).

TLR7 activation plays a role in the accumulation of autoreactive B cells and the production of autoantibodies in autoimmune diseases. One of the consequences of aberrant TLR7 activation is the accumulation of autoreactive B cells, or age-associated B cells (ABCs). ABCs are B cells that recognize self-antigens and have the potential to produce autoantibodies, which can target and damage the body's own tissues. Wang et al., Nature Communications, (2018) 9:1758.

In order to verify that the anti-BCMA/CD19 CAR-T cells also have the ability to eliminate ABCs, we will prepare CAR-T cells from the peripheral blood of healthy human donors. We will also isolate autologous B cells from the PBMCs of heathy donors and induce their differentiation in vitro to obtain ABC-enriched autologous B cells which will then be used as target cells to perform cytotoxicity experiments. After co-culture for 2 to 4 hours, the cytotoxicity of the anti-BCMA/CD19 CAR-T cells derived from different donors will be assayed, compared with control T cells without CAR transduction.

### In Vitro ABC Differentiation

PBMCs from healthy donors will be isolated by gradient centrifugation using Ficoll and cryopreserved. On the day of ABC differentiation, pan B cells will be first isolated from thawed PBMC by human B cell isolation kit (Miltenyi Biotec; negative selection, e.g., non-B cells will be labeled and depleted) according to the manufacturer's instructions. B cells will then be seeded in 96-well plates with 200 μl RPMI complete medium and stimulated with TLR7 ligand R848, CD40L, BAFF, IL-2, Goat Anti-Human IgA+IgG+IgM (H+L), IL-21, and IFN-γ for 3 days. Cell medium will be exchanged every day by replenishing with the complete medium and stimulation cocktail. The induction of ABCs will be confirmed by FACS analysis. Antibodies for FACS staining will include live/dead dye, anti-human CD19, CD38, CD27, IgD, CD11c, CD21, and T-bet.

### Cytotoxicity Assay

After differentiation, the ABC-enriched B cells will be cocultured with the anti-BCMA/CD19 CAR or non-transduced (NT) T cells at the desired E:T ratios. After 24 hours, cells will be stained with the LIVE/DEAD Fixable Aqua Dead Cell Stain (Invitrogen) to determine their viability, along with anti-CD19 and anti-CD3 antibodies to distinguish B and T cells. Cytotoxicity will be determined by the depletion of the percentage of viable CD19⁺ cells. The cytolysis of B cells will be calculated by the following formula: Percentage of lysis (%) (1−(viable CD19+ cell fraction of the anti-BCMA/CD19 CAR coculture/viable CD19⁺ cell fraction of UT coculture))×100. See, Lin et al., Preclinical evaluation of CD8+ anti-BCMA mRNA CAR T-cells for treatment of multiple myeloma. Leukemia. 2021, 35(3): 752-763.

### Example 5 Inhibitory Effect of Anti-BCMA/CD19 CAR-T Cells on Tumor Cells in Mice

The in vivo cytotoxicity effect of the anti-BCMA/CD19 CAR-T cells on CD19 or BCMA single-positive cells will be evaluated by mouse subcutaneous tumor model established using tumor cell lines expressing either CD19 (A549-CD19) or BCMA (MM.1S).

6-8 weeks female B-NDG mice will be subcutaneously inoculated with A549-CD19 (CD19+) or MM.1S (BCMA+) cells. When the average tumor volume reaches 100 mm³, anti-BCMA/CD19 CAR-T cells will be administered via the tail vein at the dosage of 3˜5×10⁶ CAR-T cells/mouse.

Specifically, female B-NDG (NOD.Cg-Prkdc^{scid} I12rg^{tm1Vst}/Vst) mice will be subcutaneously inoculated with 5×10⁶ A549-CD19 cells/animal. When the average tumor volume reaches about 100 mm³, 20 animals will be selected and randomly divided into 2 groups (vehicle control group vs. anti-BCMA/CD19 CAR group), with 10 animals in each group. A single dose of a vehicle control or the anti-BCMA/CD19 CAR T cells (3×10⁶CAR-T cells/animal) will be administered to the mice by tail vein injection.

To evaluate the in vivo effects of the anti-BCMA/CD19 CAR on BCMA single positive target cells, 20 female B-NDG (NOD.CB17-Prkdc^{scid}I12rg^{tm1}/Bcgen) mice will be subcutaneously inoculated with 5×10⁶ MM.1S cells/animal. When the average tumor volume reaches about 100 mm³, 15 animals will be selected and randomly divided into 3 groups (a vehicle control group vs. two anti-BCMA/CD19 CAR groups), with 5 animals in each group. Each mouse will be dosed once by tail vein injection. For anti-BCMA/CD19 CAR, the dosage will be 5×10⁶ CAR-T cells/animal.

To evaluate the in vivo anti-tumor effects of anti-BCMA/CD19 CAR, 65 female B-NDG (NOD.CB17-Prkdc^{scid}I12rg^{tm1}/Bcgen) mice will be subcutaneous inoculated with 1×10⁶ K562-CD19-BCMA cells/animal. When the average tumor volume reaches about 100 mm³, 50 animals will be selected and randomly divided into 5 groups: vehicle control group, T cell control group, anti-BCMA/CD19 CAR low-dose group (1×10⁶ CAR-T cells/mouse), medium-dose group (5×10⁶ CAR-T cells/mouse) and high-dose group (10×10⁶ CAR-T cells/mouse). The T cell control group will be injected with non-transduced T cells from the same donor as anti-BCMA/CD19 CAR, and the dose will be consistent with the total T cell number in the anti-BCMA/CD19 CAR high-dose group. Each mouse will be dosed once by tail vein injection.

### Example 6 Activity of Anti-BCMA/CD19 CAR Against Autologous B Cells from SLE Patients

To study CAR-T therapies for the treatment of autoimmune diseases, such as SLE, we will evaluate the efficiency of the CAR-T cells to deplete autoreactive B cells. We will also study the efficacy of the CAR-T cells on remission and survival of a lupus model.

Efficiency of Anti-BCMA/CD19 CAR to Eliminate Pan B Cells from Lupus Patients In Vitro

10˜15 mL of peripheral blood samples from eight patients with SLE will be collected. The patients will have different activity and autoantibody profile, display different organ damage (patients with lupus nephritis will be preferable), and undergo different treatment, to represent the heterogenous nature of lupus patients. Patients who recently received B cell depleting antibodies will be excluded.

For each sample, part of the blood will be used to isolate T cells for CAR-T production, and the remaining blood will be used to isolate pan B cells as target for a cytolytic assay. T cells isolated from SLE patients will be transduced by lentiviral vectors encoding anti-BCMA/CD19 CAR and test for CAR expression.

The anti-BCMA/CD19 CAR-T cells generated from patient samples, or non-transduced (NT) T cells, will be co-cultured with target cell lines expressing CD19 or/and BCMA, and the IFN-γ levels will be assessed by ELISA (enzyme-linked immunosorbent assay).

Isolated pan B cells isolated from 8 patient samples will be co-cultured with autologous anti-BCMA/CD19 CAR-T cells, or non-transduced (NT) T cells, at the desired E:T (effector to target) ratios. After 24 hours, co-culture supernatants will be collected for ELISA to assess the IFN-γ levels. Cytotoxicity will be determined by fluorescence-activated cell sorting (FACS) and calculation of the depletion of the percentage of viable CD19+ pan B cells. The cytolysis of B cells will be calculated by the following formula: Percentage of lysis (%)=(1−(viable CD19+ cell fraction of the anti-BCMA/CD19 CAR-T coculture/viable CD19+ cell fraction of UT coculture))×100.

Efficiency of the CAR-T to Eliminate ABCs from Lupus Patients In Vitro

The efficiency of the CAR-T to eliminate ABCs, the essential subset of pathogenic B cells, from lupus patients in vitro will be studied.

Blood samples or PBMCs from lupus patients will be processed for ABCs differentiation and CAR-T production as well as functional analysis.

The study will confirm the efficiency of the CAR-T cells to deplete ABCs from lupus patients in the in vitro setting.

### Efficiency of the CAR-T to Deplete B Cells and the Therapeutic Efficacy In Vivo

The efficiency of the CAR-T to deplete B cells and its therapeutic efficacy will be evaluated in vivo with a humanized mouse model of SLE. CD34⁺ stem cell humanized mice will be obtained. 2 or more mice will be sacrificed to collect spleens with aseptic technique. T cells will then be isolated from the spleens for CAR-T production. The remaining mice will be used to induce the onset of lupus disease, and upon successful induction, mice will be divided into groups to receive CAR-T or control treatment (for example, non-transduced T cells). Blood samples will be obtained from the mice periodically to monitor the persistence of CAR-T cells, as well as efficiency of B cell depletion (including ABCs) by FACS. The sera samples will be used to measure the titers of various autoantibodies. Urine samples will also be routinely collected to measure the levels of proteinuria. At the end of the study, or in case an animal dies early (presumably in control group), tissues will be collected for histology, for example, to examine the deposition of immune complex in the kidney and the severity of nephritis. The presence of B cells or plasma cells in diseased tissue will also be examined. Survival curves will be generated to compare the effect of CAR-T versus control treatment.

The structures of the anti-BCMA/CD19 CARs, TB19-1 to TB19-4 and TB19-L1 to TB19-L4, are shown in Table 1.

TABLE 1

| Anti- BCMA/ | | |
|---|---|---|
| CD19 CAR | scFv V_{H}/V_{L} order | CAR structure |
| TN-B20-19-1 | B20(V_{L}-V_{H}) - | SP - B20 V_{L} - linker 1 - B20 V_{H} - linker 2 - |
| (TB19-1) | FMC63(V_{L}-V_{H}) | FMC63 V_{L} - linker 3 - FMC63 V_{H} - IgG4 hinge - |
| | | CD28 TM - 4-1BB - CD3z |
| TN-B20-19-2 | B20(V_{H}-V_{L}) - | SP - B20 V_{H} - linker 1 - B20 V_{L} - linker 2 - |
| (TB19-2) | FMC63(V_{L}-V_{H}) | FMC63 V_{L} - linker 3 - FMC63 V_{H} - IgG4 hinge - |
| | | CD28 TM - 4-1BB - CD3z |
| TN-B20-19-3 | B20(V_{H}-V_{L}) - | SP - B20 V_{H} - linker 1 - B20 V_{L} - linker 2 - |
| (TB19-3) | FMC63(V_{H}-V_{L}) | FMC63 V_{H} - linker 3 - FMC63 V_{L} - IgG4 hinge - |
| | | CD28 TM - 4-1BB - CD3z |
| TN-B20-19-4 | B20(V_{L}-V_{H}) - | SP - B20 V_{L} - linker 1 - B20 V_{H} - linker 2 - |
| (TB19-4) | FMC63(V_{H}-V_{L}) | FMC63 V_{H} - linker 3 - FMC63 V_{L} - IgG4 hinge - |
| | | CD28 TM - 4-1BB - CD3z |
| TN-B20-19-L1 | B20(V_{L}-V_{H}) - | SP - B20 V_{L} - linker 1 - B20 V_{H} - linker 2 - |
| (TB19-L1) | FMC63(V_{L}-V_{H}) | FMC63 V_{L} - linker 3 - FMC63 V_{H} - CD8a hinge - |
| | | CD8 TM - 4-1BB - CD3z |
| TN-B20-19-L2 | B20(V_{H}-V_{L}) - | SP - B20 V_{H} - linker 1 - B20 V_{L} - linker 2 - |
| (TB19-L2) | FMC63(V_{L}-V_{H}) | FMC63 V_{L} - linker 3 - FMC63 V_{H} - CD8a hinge - |
| | | CD8 TM - 4-1BB - CD3z |
| TN-B20-19-L3 | B20(V_{H}-V_{L}) - | SP - B20 V_{H} - linker 1 - B20 V_{L} - linker 2 - |
| (TB19-L3) | FMC63(V_{H}-V_{L}) | FMC63 V_{H} - linker 3 - FMC63 V_{L} - CD8a hinge - |
| | | CD8 TM - 4-1BB - CD3z |
| TN-B20-19-L4 | B20(V_{L}-V_{H}) - | SP - B20 V_{L} - linker 1 - B20 V_{H} - linker 2 - |
| (TB 19-L4) | FMC63(V_{H}-V_{L}) | FMC63 V_{H} - linker 3 - FMC63 V_{L} - CD8a hinge - |
| | | CD8 TM - 4-1BB - CD3z |

Sequences:
BCMA-20 (B20) V_{L}

| | | Residues | | |
|---|---|---|---|---|
| | | of SEQ ID | | SEQ ID |
| Region | Sequence Fragment | No: 24 | Length | No: |
| LFR1 | DIQMTQSPSSLSASVGDRVTITC | 1-23 | 23 | SEQ ID |
| | | | | No: 1 |
| CDR-L1 | RASQGISNYLN | 24-34 | 11 | SEQ ID |
| | | | | No: 2 |
| LFR2 | WYQQKPGKAPKPLIY | 35-49 | 15 | SEQ ID |
| | | | | No: 3 |
| CDR-L2 | YTSNLQS | 50-56 | 7 | SEQ ID |
| | | | | No: 4 |
| LFR3 | GVPSRFSGSGSGTDYTLTISSLQPEDFATYYC | 57-88 | 32 | SEQ ID |
| | | | | No: 5 |
| CDR-L3 | MGQTISSYT | 89-97 | 9 | SEQ ID |
| | | | | No: 6 |
| LFR4 | FGQGTKLEIK | 98-107 | 10 | SEQ ID |
| | | | | No: 7 |

BCMA-20 V_{H}

| | | Residues of | | |
|---|---|---|---|---|
| | | SEQ ID | | SEQ ID |
| Region | Sequence Fragment | No: 28 | Length | No: |
| HFR1 | EVQLVESGGGLVQPGGSLRLSCAASGFTFS | 1-30 | 30 | SEQ ID |
| | | | | No:8 |
| CDR-H1 | NFDMA | 31-35 | 5 | SEQ ID |
| | | | | No:9 |
| HFR2 | WVRQAPGKGLVWVS | 36-49 | 14 | SEQ ID |
| | | | | No:10 |
| CDR-H2 | SITTGADHAIYADSVKG | 50-66 | 17 | SEQ ID |
| | | | | No:11 |
| HFR3 | RFTISRDNAKNTLYLQMNSLRAEDTAVYYCVR | 67-98 | 32 | SEQ ID |
| | | | | No:12 |
| CDR-H3 | HGYYDGYHLFDY | 99-110 | 12 | SEQ ID |
| | | | | No:13 |
| HFR4 | WGQGTLVTVSS | 111-121 | 11 | SEQ ID |
| | | | | No:14 |

FMC63-V_{L}-CDR1: RASQDISKYLN (SEQ ID NO: 15)
FMC63-V_{L}-CDR2: HTSRLHS (SEQ ID NO: 16)
FMC63-V_{L}-CDR3: QQGNTLPYT (SEQ ID NO: 17)
FMC63-V_{H}-CDR1: DYGVS (SEQ ID NO: 18)
FMC63-V_{H}-CDR2: VIWGSETTYYNSALKS (SEQ ID NO: 19)
FMC63-V_{H}-CDR3: HYYYGGSYAMDY (SEQ ID NO: 20)
TN-B20-19-1 (TB19-1)
CD8a SP nucleic acid sequence (63 nt)
ATGGCCTTACCAGTGACCGCCTTGCTCCTGCCGCTGGCCTTGCTGCTCCACGCCG
CCAGGCCG (SEQ ID NO: 21)
CD8a SP amino acid sequence
MALPVTALLLPLALLLHAARP (SEQ ID NO: 22)
B20 V_{L} nucleic acid sequence (321 nt)
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 23)
B20 V_{L} amino acid sequence
DIQMTQSPSSLSASVGDRVTITCRASQGISNYLNWYQQKPGKAPKPLIYYTSNLQ
SGVPSRFSGSGSGTDYTLTISSLQPEDFATYYCMGQTISSYTFGQGTKLEIK (SEQ
ID NO: 24)
Linker-1 nucleic acid sequence (45 nt)
Ggtggcggtggctcgggcggtggtgggtcgggtggcggcggatct (SEQ ID NO: 25)
Linker-1 amino acid sequence
GGGGGGGGSGGGGS (SEQ ID NO: 26)
B20 V_{H} nucleic acid sequence (363 nt)
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 27)
B20 V_{H} amino acid sequence
EVQLVESGGGLVQPGGSLRLSCAASGFTFSNFDMAWVRQAPGKGLVWVSSITT
GADHAIYADSVKGRFTISRDNAKNTLYLQMNSLRAEDTAVYYCVRHGYYDGYH
LFDYWGQGTLVTVSS (SEQ ID NO: 28)
Linker-2 nucleic acid sequence (15 nt)
GGAGGTGGTGGATCC (SEQ ID NO: 29)
Linker-2 amino acid sequence
GGGGS (SEQ ID NO: 30)
FMC63 V_{L} nucleic acid sequence (321 nt)
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgattaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 31)
FMC63 V_{L} amino acid sequence
DIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEIT (SEQ
ID NO: 32)
Linker-3 nucleic acid sequence (45 nt)
Ggtggcggtggctcgggcggtggtgggtcgggtggcggcggatct (SEQ ID NO: 33)
Linker-3 amino acid sequence
GGGGSGGGGSGGGGS (SEQ ID NO: 34)
FMC63 V_{H} nucleic acid sequence (360 nt)
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 35)
FMC63 V_{H} amino acid sequence
EVKLQESGPGLVAPSQSLSVTCTVSGVSLPDYGVSWIRQPPRKGLEWLGVIWGS
ETTYYNSALKSRLTIIKDNSKSQVFLKMNSLQTDDTAIYYCAKHYYYGGSYAMD
YWGQGTSVTVSS (SEQ ID NO: 36)
IgG4 hinge nucleic acid sequence (36 nt)
GAGAGCAAGTACGGACCGCCCTGCCCCCCTTGCCCT (SEQ ID NO: 37)
IgG4 hinge amino acid sequence
ESKYGPPCPPCP (SEQ ID NO: 38)
CD28 TM (84 nt)
ATGTTCTGGGTGCTGGTGGTGGTCGGAGGCGTGCTGGCCTGCTACAGCCTGCTGG
TCACCGTGGCCTTCATCATCTTTTGGGTG (SEQ ID NO: 39)
CD28 TM amino acid sequence
MFWVLVVVGGVLACYSLLVTVAFIIFWV (SEQ ID NO: 40)
4-1BB co-stimulatory region nucleic acid sequence (126 nt)
Aaacggggcagaaagaaactcctgtatatattcaaacaaccatttatgagaccagtacaaactactcaagaggaagatggctgtag
ctgccgatttccagaagaagaagaaggaggatgtgaactg (SEQ ID NO: 41)
4-1BB co-stimulatory region amino acid sequence
KRGRKKLLYIFKQPFMRPVQTTQEEDGCSCRFPEEEEGGCEL (SEQ ID NO: 42)
CD3z cytoplasmic signaling domain nucleic acid sequence (336 nt)
Agagtgaagttcagcaggagcgcagacgcccccgcgtaccagcagggccagaaccagctctataacgagctcaatctaggacg
aagagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagggg
caaggggcacgatggcctttaccagggtctcagtacagccaccaaggacacctacgacgcccttcacatgcaggccctgccccct
cgctaa (SEQ ID NO: 43)
CD3z cytoplasmic signaling domain amino acid sequence
RVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDAL
HMQALPPR (SEQ ID NO: 44)
TB19-1 nucleic acid sequence (2118 nt):
ATGGCCTTACCAGTGACCGCCTTGCTCCTGCCGCTGGCCTTGCTGCTCCACGCCG
CCAGGCCGgacatccagatgacccagtccccctcctccctgtccgcctccgtgggcgaccgggtgaccatcacctgccgg
gcctcccagggcatctccaactacctgaactggtaccagcagaagcccggcaaggcccccaagcccctgatctactacacctcca
acctgcagtccggcgtgccctcccggttctccggctccggctccggcaccgactacaccctgaccatctcctccctgcagcccgag
gacttcgccacctactactgcatgggccagaccatctcctcctacaccttcggccagggcaccaagctggagatcaagggtggcg
gtggctcgggcggtggtgggtcgggtggcggcggatctgaggtgcagctggtggagtccggcggcggcctggtgcagcccgg
cggctccctgcggctgtcctgcgccgcctccggcttcaccttctccaacttcgacatggcctgggtgcggcaggcccccggcaag
ggcctggtgtgggtgtcctccatcaccaccggcgccgaccacgccatctacgccgactccgtgaagggccggttcaccatctccc
gggacaacgccaagaacaccctgtacctgcagatgaactccctgcgggccgaggacaccgccgtgtactactgcgtgcggcacg
gctactacgacggctaccacctgttcgactactggggccagggcaccctggtgaccgtgtcctccGGAGGTGGTGGAT
CCgacatccagatgacacagactacatcctccctgtctgcctctctgggagacagagtcaccatcagttgcagggcaagtcagga
cattagtaaatatttaaattggtatcagcagaaaccagatggaactgttaaactcctgatctaccatacatcaagattacactcaggagt
cccatcaaggttcagtggcagtgggtctggaacagattattctctcaccattagcaacctggagcaagaagatattgccacttactttt
gccaacagggtaatacgcttccgtacacgttcggaggggggaccaagctggagatcacaggtggcggtggctcgggcggtggt
gggtcgggtggcggcggatctgaggtgaaactgcaggagtcaggacctggcctggtggcgccctcacagagcctgtccgtcaca
tgcactgtctcaggggtctcattacccgactatggtgtaagctggattcgccagcctccacgaaagggtctggagtggctgggagta
atatggggtagtgaaaccacatactataattcagctctcaaatccagactgaccatcatcaaggacaactccaagagccaagttttctt
aaaaatgaacagtctgcaaactgatgacacagccatttactactgtgccaaacattattactacggtggtagctatgctatggactact
ggggccaaggaacctcagtcaccgtctcctcaGAGAGCAAGTACGGACCGCCCTGCCCCCCTTGCC
CTATGTTCTGGGTGCTGGTGGTGGTCGGAGGCGTGCTGGCCTGCTACAGCCTGCT
GGTCACCGTGGCCTTCATCATCTTTTGGGTGAAACGGGGCAGAAAGAAACTCCTG
TATATATTCAAACAACCATTTATGAGACCAGTACAAACTACTCAAGAGGAAGAT
GGCTGTAGCTGCCGATTTCCAGAAGAAGAAGAAGGAGGATGTGAACTGCGGGTG
AAGTTCAGCAGAAGCGCCGACGCCCCTGCCTACCAGCAGGGCCAGAATCAGCTG
TACAACGAGCTGAACCTGGGCAGAAGGGAAGAGTACGACGTCCTGGATAAGCGG
AGAGGCCGGGACCCTGAGATGGGCGGCAAGCCTCGGCGGAAGAACCCCCAGGA
AGGCCTGTATAACGAACTGCAGAAAGACAAGATGGCCGAGGCCTACAGCGAGAT
CGGCATGAAGGGCGAGCGGAGGCGGGGCAAGGGCCACGACGGCCTGTATCAGG
GCCTGTCCACCGCCACCAAGGATACCTACGACGCCCTGCACATGCAGGCCCTGCC
CCCAAGGTAA (SEQ ID NO: 45)
TB19-1 amino acid sequence:
MALPVTALLLPLALLLHAARPDIQMTQSPSSLSASVGDRVTITCRASQGISNYLN
WYQQKPGKAPKPLIYYTSNLQSGVPSRFSGSGSGTDYTLTISSLQPEDFATYYCM
GQTISSYTFGQGTKLEIKGGGGSGGGGSGGGGSEVQLVESGGGLVQPGGSLRLS
CAASGFTFSNFDMAWVRQAPGKGLVWVSSITTGADHAIYADSVKGRFTISRDNA
KNTLYLQMNSLRAEDTAVYYCVRHGYYDGYHLFDYWGQGTLVTVSSGGGGSD
IQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIYYCAK
HYYYGGSYAMDYWGQGTSVTVSSESKYGPPCPPCPMFWVLVVVGGVLACYSL
LVTVAFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALH
MQALPPR (SEQ ID NO: 46)
TN-B20-19-2 (TB19-2)
CD8a SP nucleic acid sequence
ATGGCCTTACCAGTGACCGCCTTGCTCCTGCCGCTGGCCTTGCTGCTCCACGCCG
CCAGGCCG (SEQ ID NO: 47)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 48)
Linker-1
Ggtggcggtggctcgggcggtggtgggtcgggtggcggcggatct (SEQ ID NO: 49)
B20 V_{L}
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttgccacc
tactactgcatgggccagaccatctcctcctacaccttcggccagggcaccaagctggagatcaag (SEQ ID NO: 50)
Linker-2
GGAGGTGGTGGATCC (SEQ ID NO: 51)
FMC63 V_{L}
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgattaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 52)
Linker-3
Ggtggcggtggctcgggcggtggtgggtcgggtggcggcggatct (SEQ ID NO: 53)
FMC63 V_{H}
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 54)
Hinge
GAGAGCAAGTACGGACCGCCCTGCCCCCCTTGCCCT (SEQ ID NO: 55)
CD28 TM
ATGTTCTGGGTGCTGGTGGTGGTCGGAGGCGTGCTGGCCTGCTACAGCCTGCTGG
TCACCGTGGCCTTCATCATCTTTTGGGTG (SEQ ID NO: 56)
4-1BB co-stimulatory region
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG (SEQ ID NO: 57)
CD3z cytoplasmic signaling domain
CGGGTGAAGTTCAGCAGAAGCGCCGACGCCCCTGCCTACCAGCAGGGCCAGAAT
CAGCTGTACAACGAGCTGAACCTGGGCAGAAGGGAAGAGTACGACGTCCTGGAT
AAGCGGAGAGGCCGGGACCCTGAGATGGGCGGCAAGCCTCGGCGGAAGAACCC
CCAGGAAGGCCTGTATAACGAACTGCAGAAAGACAAGATGGCCGAGGCCTACAG
CGAGATCGGCATGAAGGGCGAGCGGAGGCGGGGCAAGGGCCACGACGGCCTGT
ATCAGGGCCTGTCCACCGCCACCAAGGATACCTACGACGCCCTGCACATGCAGG
CCCTGCCCCCAAGG (SEQ ID NO: 58)
TB19-2 (2118 nt)
ATGGCCTTACCAGTGACCGCCTTGCTCCTGCCGCTGGCCTTGCTGCTCCACGCCG
CCAGGCCGGaggtgcagctggtggagtccggcggcggcctggtgcagcccggcggctccctgcggctgtcctgcgccg
cctccggcttcaccttctccaacttcgacatggcctgggtgcggcaggcccccggcaagggcctggtgtgggtgtcctccatcacc
accggcgccgaccacgccatctacgccgactccgtgaagggccggttcaccatctcccgggacaacgccaagaacaccctgtac
ctgcagatgaactccctgcgggccgaggacaccgccgtgtactactgcgtgcggcacggctactacgacggctaccacctgttcg
actactggggccagggcaccctggtgaccgtgtcctccggtggcggtggctcgggcggtggtgggtcgggtggcggcggatctg
acatccagatgacccagtccccctcctccctgtccgcctccgtgggcgaccgggtgaccatcacctgccgggcctcccagggcat
ctccaactacctgaactggtaccagcagaagcccggcaaggcccccaagcccctgatctactacacctccaacctgcagtccggc
gtgccctcccggttctccggctccggctccggcaccgactacaccctgaccatctcctccctgcagcccgaggacttcgccaccta
ctactgcatgggccagaccatctcctcctacaccttcggccagggcaccaagctggagatcaagGGAGGTGGTGGAT
CCgacatccagatgacacagactacatcctccctgtctgcctctctgggagacagagtcaccatcagttgcagggcaagtcagga
cattagtaaatatttaaattggtatcagcagaaaccagatggaactgttaaactcctgatctaccatacatcaagattacactcaggagt
cccatcaaggttcagtggcagtgggtctggaacagattattctctcaccattagcaacctggagcaagaagatattgccacttactttt
gccaacagggtaatacgcttccgtacacgttcggaggggggaccaagctggagatcacaggtggcggtggctcgggcggtggt
gggtcgggtggcggcggatctgaggtgaaactgcaggagtcaggacctggcctggtggcgccctcacagagcctgtccgtcaca
tgcactgtctcaggggtctcattacccgactatggtgtaagctggattcgccagcctccacgaaagggtctggagtggctgggagta
atatggggtagtgaaaccacatactataattcagctctcaaatccagactgaccatcatcaaggacaactccaagagccaagttttctt
aaaaatgaacagtctgcaaactgatgacacagccatttactactgtgccaaacattattactacggtggtagctatgctatggactact
ggggccaaggaacctcagtcaccgtctcctcaGAGAGCAAGTACGGACCGCCCTGCCCCCCTTGCC
CTATGTTCTGGGTGCTGGTGGTGGTCGGAGGCGTGCTGGCCTGCTACAGCCTGCT
GGTCACCGTGGCCTTCATCATCTTTTGGGTGAAACGGGGCAGAAAGAAACTCCTG
TATATATTCAAACAACCATTTATGAGACCAGTACAAACTACTCAAGAGGAAGAT
GGCTGTAGCTGCCGATTTCCAGAAGAAGAAGAAGGAGGATGTGAACTGCGGGTG
AAGTTCAGCAGAAGCGCCGACGCCCCTGCCTACCAGCAGGGCCAGAATCAGCTG
TACAACGAGCTGAACCTGGGCAGAAGGGAAGAGTACGACGTCCTGGATAAGCGG
AGAGGCCGGGACCCTGAGATGGGCGGCAAGCCTCGGCGGAAGAACCCCCAGGA
AGGCCTGTATAACGAACTGCAGAAAGACAAGATGGCCGAGGCCTACAGCGAGAT
CGGCATGAAGGGCGAGCGGAGGCGGGGCAAGGGCCACGACGGCCTGTATCAGG
GCCTGTCCACCGCCACCAAGGATACCTACGACGCCCTGCACATGCAGGCCCTGCC
CCCAAGGTAA (SEQ ID NO: 59)
TB19-2 amino acid sequence
MALPVTALLLPLALLLHAARPEVQLVESGGGLVQPGGSLRLSCAASGFTFSNFD
MAWVRQAPGKGLVWVSSITTGADHAIYADSVKGRFTISRDNAKNTLYLQMNSL
RAEDTAVYYCVRHGYYDGYHLFDYWGQGTLVTVSSGGGGSGGGGSGGGGSDI
QMTQSPSSLSASVGDRVTITCRASQGISNYLNWYQQKPGKAPKPLIYYTSNLQSG
VPSRFSGSGSGTDYTLTISSLQPEDFATYYCMGQTISSYTFGQGTKLEIKGGGGS
DIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGG
GSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCA
KHYYYGGSYAMDYWGQGTSVTVSSESKYGPPCPPCPMFWVLVVVGGVLACYS
LLVTVAFIIFWVKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDAL
HMQALPPR (SEQ ID NO: 60)
TN-B20-19-3 (TB19-3)
CD8a SP
ATGGCCTTACCAGTGACCGCCTTGCTCCTGCCGCTGGCCTTGCTGCTCCACGCCG
CCAGGCCG (SEQ ID NO: 61)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 62)
Linker-1
Ggtggcggtggctcgggcggtggtgggtcgggtggcggcggatct (SEQ ID NO: 63)
B20 V_{L}
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 64)
Linker-2
GGAGGTGGTGGATCC (SEQ ID NO: 65)
FMC63 V_{H}
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 66)
Linker-3
Ggtggcggtggctcgggcggtggtgggtcgggtggcggcggatct (SEQ ID NO: 67)
FMC63 V_{L}
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 68)
Hinge
GAGAGCAAGTACGGACCGCCCTGCCCCCCTTGCCCT (SEQ ID NO: 69)
CD28 TM
ATGTTCTGGGTGCTGGTGGTGGTCGGAGGCGTGCTGGCCTGCTACAGCCTGCTGG
TCACCGTGGCCTTCATCATCTTTTGGGTG (SEQ ID NO: 70)
4-1BB co-stimulatory region
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG (SEQ ID NO: 71)
CD3z cytoplasmic signaling domain
CGGGTGAAGTTCAGCAGAAGCGCCGACGCCCCTGCCTACCAGCAGGGCCAGAAT
CAGCTGTACAACGAGCTGAACCTGGGCAGAAGGGAAGAGTACGACGTCCTGGAT
AAGCGGAGAGGCCGGGACCCTGAGATGGGCGGCAAGCCTCGGCGGAAGAACCC
CCAGGAAGGCCTGTATAACGAACTGCAGAAAGACAAGATGGCCGAGGCCTACAG
CGAGATCGGCATGAAGGGCGAGCGGAGGCGGGGCAAGGGCCACGACGGCCTGT
ATCAGGGCCTGTCCACCGCCACCAAGGATACCTACGACGCCCTGCACATGCAGG
CCCTGCCCCCAAGG (SEQ ID NO: 72)
TB19-3 (2118 nt)
ATGGCCTTACCAGTGACCGCCTTGCTCCTGCCGCTGGCCTTGCTGCTCCACGCCG
CCAGGCCGGaggtgcagctggtggagtccggcggcggcctggtgcagcccggcggctccctgcggctgtcctgcgccg
cctccggcttcaccttctccaacttcgacatggcctgggtgcggcaggcccccggcaagggcctggtgtgggtgtcctccatcacc
accggcgccgaccacgccatctacgccgactccgtgaagggccggttcaccatctcccgggacaacgccaagaacaccctgtac
ctgcagatgaactccctgcgggccgaggacaccgccgtgtactactgcgtgcggcacggctactacgacggctaccacctgttcg
actactggggccagggcaccctggtgaccgtgtcctccggtggcggtggctcgggcggtggtgggtcgggtggcggcggatctg
acatccagatgacccagtccccctcctccctgtccgcctccgtgggcgaccgggtgaccatcacctgccgggcctcccagggcat
ctccaactacctgaactggtaccagcagaagcccggcaaggcccccaagcccctgatctactacacctccaacctgcagtccggc
gtgccctcccggttctccggctccggctccggcaccgactacaccctgaccatctcctccctgcagcccgaggacttcgccaccta
ctactgcatgggccagaccatctcctcctacaccttcggccagggcaccaagctggagatcaagGGAGGTGGTGGAT
CCgaggtgaaactgcaggagtcaggacctggcctggtggcgccctcacagagcctgtccgtcacatgcactgtctcaggggtct
cattacccgactatggtgtaagctggattcgccagcctccacgaaagggtctggagtggctgggagtaatatggggtagtgaaacc
acatactataattcagctctcaaatccagactgaccatcatcaaggacaactccaagagccaagttttcttaaaaatgaacagtctgca
aactgatgacacagccatttactactgtgccaaacattattactacggtggtagctatgctatggactactggggccaaggaacctca
gtcaccgtctcctcaggtggcggtggctcgggcggtggtgggtcgggtggcggcggatctgacatccagatgacacagactacat
cctccctgtctgcctctctgggagacagagtcaccatcagttgcagggcaagtcaggacattagtaaatatttaaattggtatcagca
gaaaccagatggaactgttaaactcctgatctaccatacatcaagattacactcaggagtcccatcaaggttcagtggcagtgggtct
ggaacagattattctctcaccattagcaacctggagcaagaagatattgccacttacttttgccaacagggtaatacgcttccgtacac
gttcggaggggggaccaagctggagatcacaGAGAGCAAGTACGGACCGCCCTGCCCCCCTTGCC
CTATGTTCTGGGTGCTGGTGGTGGTCGGAGGCGTGCTGGCCTGCTACAGCCTGCT
GGTCACCGTGGCCTTCATCATCTTTTGGGTGAAACGGGGCAGAAAGAAACTCCTG
TATATATTCAAACAACCATTTATGAGACCAGTACAAACTACTCAAGAGGAAGAT
GGCTGTAGCTGCCGATTTCCAGAAGAAGAAGAAGGAGGATGTGAACTGCGGGTG
AAGTTCAGCAGAAGCGCCGACGCCCCTGCCTACCAGCAGGGCCAGAATCAGCTG
TACAACGAGCTGAACCTGGGCAGAAGGGAAGAGTACGACGTCCTGGATAAGCGG
AGAGGCCGGGACCCTGAGATGGGCGGCAAGCCTCGGCGGAAGAACCCCCAGGA
AGGCCTGTATAACGAACTGCAGAAAGACAAGATGGCCGAGGCCTACAGCGAGAT
CGGCATGAAGGGCGAGCGGAGGCGGGGCAAGGGCCACGACGGCCTGTATCAGG
GCCTGTCCACCGCCACCAAGGATACCTACGACGCCCTGCACATGCAGGCCCTGCC
CCCAAGGTAA (SEQ ID NO: 73)
TB19-3 amino acid sequence
MALPVTALLLPLALLLHAARPEVOLVESGGGLVQPGGSLRLSCAASGFTFSNFD
MAWVRQAPGKGLVWVSSITTGADHAIYADSVKGRFTISRDNAKNTLYLQMNSL
RAEDTAVYYCVRHGYYDGYHLFDYWGQGTLVTVSSGGGGSGGGGSGGGGSDI
QMTQSPSSLSASVGDRVTITCRASQGISNYLNWYQQKPGKAPKPLIYYTSNLQSG
VPSRFSGSGSGTDYTLTISSLQPEDFATYYCMGQTISSYTFGQGTKLEIKGGGGS
EVKLQESGPGLVAPSQSLSVTCTVSGVSLPDYGVSWIRQPPRKGLEWLGVIWGS
ETTYYNSALKSRLTIIKDNSKSQVFLKMNSLQTDDTAIYYCAKHYYYGGSYAMD
YWGQGTSVTVSSGGGGSGGGGSGGGGSDIQMTQTTSSLSASLGDRVTISCRASQ
DISKYLNWYQQKPDGTVKLLIYHTSRLHSGVPSRFSGSGSGTDYSLTISNLEQED
IATYFCQQGNTLPYTFGGGTKLEITESKYGPPCPPCPMFWVLVVVGGVLACYSL
LVTVAFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALH
MQALPPR (SEQ ID NO: 74)
TN-B20-19-4 (TB19-4)
CD8a SP
ATGGCCTTACCAGTGACCGCCTTGCTCCTGCCGCTGGCCTTGCTGCTCCACGCCG
CCAGGCCG (SEQ ID NO: 75)
B20 V_{L}
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 76)
Linker-1
Ggtggcggtggctcgggcggtggtgggtcgggtggcggcggatct (SEQ ID NO: 77)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 78)
Linker-2
GGAGGTGGTGGATCC (SEQ ID NO: 79)
FMC63 V_{H}
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 80)
Linker-3
Ggtggcggtggctcgggcggtggtgggtcgggtggcggcggatct (SEQ ID NO: 81)
FMC63 V_{L}
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 82)
Hinge
GAGAGCAAGTACGGACCGCCCTGCCCCCCTTGCCCT (SEQ ID NO: 83)
CD28 TM
ATGTTCTGGGTGCTGGTGGTGGTCGGAGGCGTGCTGGCCTGCTACAGCCTGCTGG
TCACCGTGGCCTTCATCATCTTTTGGGTG (SEQ ID NO: 84)
4-1BB co-stimulatory region
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG (SEQ ID NO: 85)
CD3z cytoplasmic signaling domain
CGGGTGAAGTTCAGCAGAAGCGCCGACGCCCCTGCCTACCAGCAGGGCCAGAAT
CAGCTGTACAACGAGCTGAACCTGGGCAGAAGGGAAGAGTACGACGTCCTGGAT
AAGCGGAGAGGCCGGGACCCTGAGATGGGCGGCAAGCCTCGGCGGAAGAACCC
CCAGGAAGGCCTGTATAACGAACTGCAGAAAGACAAGATGGCCGAGGCCTACAG
CGAGATCGGCATGAAGGGCGAGCGGAGGCGGGGCAAGGGCCACGACGGCCTGT
ATCAGGGCCTGTCCACCGCCACCAAGGATACCTACGACGCCCTGCACATGCAGG
CCCTGCCCCCAAGG (SEQ ID NO: 86)
TB19-4 (2118 nt)
ATGGCCTTACCAGTGACCGCCTTGCTCCTGCCGCTGGCCTTGCTGCTCCACGCCG
CCAGGCCGgacatccagatgacccagtccccctcctccctgtccgcctccgtgggcgaccgggtgaccatcacctgccgg
gcctcccagggcatctccaactacctgaactggtaccagcagaagcccggcaaggcccccaagcccctgatctactacacctcca
acctgcagtccggcgtgccctcccggttctccggctccggctccggcaccgactacaccctgaccatctcctccctgcagcccgag
gacttcgccacctactactgcatgggccagaccatctcctcctacaccttcggccagggcaccaagctggagatcaagggtggcg
gtggctcgggcggtggtgggtcgggtggcggcggatctGaggtgcagctggtggagtccggcggcggcctggtgcagcccgg
cggctccctgcggctgtcctgcgccgcctccggcttcaccttctccaacttcgacatggcctgggtgcggcaggcccccggcaag
ggcctggtgtgggtgtcctccatcaccaccggcgccgaccacgccatctacgccgactccgtgaagggccggttcaccatctccc
gggacaacgccaagaacaccctgtacctgcagatgaactccctgcgggccgaggacaccgccgtgtactactgcgtgcggcacg
gctactacgacggctaccacctgttcgactactggggccagggcaccctggtgaccgtgtcctccGGAGGTGGTGGAT
CCgaggtgaaactgcaggagtcaggacctggcctggtggcgccctcacagagcctgtccgtcacatgcactgtctcaggggtct
cattacccgactatggtgtaagctggattcgccagcctccacgaaagggtctggagtggctgggagtaatatggggtagtgaaacc
acatactataattcagctctcaaatccagactgaccatcatcaaggacaactccaagagccaagttttcttaaaaatgaacagtctgca
aactgatgacacagccatttactactgtgccaaacattattactacggtggtagctatgctatggactactggggccaaggaacctca
gtcaccgtctcctcaggtggcggtggctcgggcggtggtgggtcgggtggcggcggatctgacatccagatgacacagactacat
cctccctgtctgcctctctgggagacagagtcaccatcagttgcagggcaagtcaggacattagtaaatatttaaattggtatcagca
gaaaccagatggaactgttaaactcctgatctaccatacatcaagattacactcaggagtcccatcaaggttcagtggcagtgggtct
ggaacagattattctctcaccattagcaacctggagcaagaagatattgccacttacttttgccaacagggtaatacgcttccgtacac
gttcggaggggggaccaagctggagatcacaGAGAGCAAGTACGGACCGCCCTGCCCCCCTTGCC
CTATGTTCTGGGTGCTGGTGGTGGTCGGAGGCGTGCTGGCCTGCTACAGCCTGCT
GGTCACCGTGGCCTTCATCATCTTTTGGGTGAAACGGGGCAGAAAGAAACTCCTG
TATATATTCAAACAACCATTTATGAGACCAGTACAAACTACTCAAGAGGAAGAT
GGCTGTAGCTGCCGATTTCCAGAAGAAGAAGAAGGAGGATGTGAACTGCGGGTG
AAGTTCAGCAGAAGCGCCGACGCCCCTGCCTACCAGCAGGGCCAGAATCAGCTG
TACAACGAGCTGAACCTGGGCAGAAGGGAAGAGTACGACGTCCTGGATAAGCGG
AGAGGCCGGGACCCTGAGATGGGCGGCAAGCCTCGGCGGAAGAACCCCCAGGA
AGGCCTGTATAACGAACTGCAGAAAGACAAGATGGCCGAGGCCTACAGCGAGAT
CGGCATGAAGGGCGAGCGGAGGCGGGGCAAGGGCCACGACGGCCTGTATCAGG
GCCTGTCCACCGCCACCAAGGATACCTACGACGCCCTGCACATGCAGGCCCTGCC
CCCAAGGTAA (SEQ ID NO: 87)
TB19-4 amino acid sequence
MALPVTALLLPLALLLHAARPDIQMTQSPSSLSASVGDRVTITCRASQGISNYLN
WYQQKPGKAPKPLIYYTSNLQSGVPSRFSGSGSGTDYTLTISSLQPEDFATYYCM
GQTISSYTFGQGTKLEIKGGGGSGGGGSGGGGSEVQLVESGGGLVQPGGSLRLS
CAASGFTFSNFDMAWVRQAPGKGLVWVSSITTGADHAIYADSVKGRFTISRDNA
KNTLYLQMNSLRAEDTAVYYCVRHGYYDGYHLFDYWGQGTLVTVSSGGGGSE
VKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDY
WGQGTSVTVSSGGGGSGGGGSGGGGSDIQMTQTTSSLSASLGDRVTISCRASQD
ISKYLNWYQQKPDGTVKLLIYHTSRLHSGVPSRFSGSGSGTDYSLTISNLEQEDI
ATYFCQQGNTLPYTFGGGTKLEITESKYGPPCPPCPMFWVLVVVGGVLACYSL
LVTVAFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALH
MQALPPR (SEQ ID NO: 88)
TN-B20-19-L1 (TB19-L1)
CD8a SP (63 nt)
ATGGCCTTACCAGTGACCGCCTTGCTCCTGCCGCTGGCCTTGCTGCTCCACGCCG
CCAGGCCG (SEQ ID NO: 89)
B20 V_{L} (321 nt)
Gacatccagatgacccagtccccctcctccctgtccgcctccgtggggaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 90)
Linker-1 (45 nt)
Ggtggcggtggctcgggcggtggtgggtcgggtggcggcggatct (SEQ ID NO: 91)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 92)
Linker-2 (15 nt)
GGAGGTGGTGGATCC (SEQ ID NO: 93)
FMC63 V_{L} (321 nt)
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 94)
Linker-3 (45 nt)
Ggtggcggtggctcgggcggtggtgggtcgggtggcggoggatct (SEQ ID NO: 95)
FMC63 V_{H} (360 nt)
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 96)
CD8a hinge (165 nt)
Ttcgtgccggtcttcctgccagcgaagcccaccacgacgccagcgccgcgaccaccaacaccggcgcccaccatcgcgtcgca
gcccctgtccctgcgcccagaggcgtgccggccagcggcggggggcgcagtgcacacgagggggctggacttcgcctgtgat
(SEQ ID NO: 97)
CD8a hinge amino acid sequence
FVPVFLPAKPTTTPAPRPPTPAPTIASQPLSLRPEACRPAAGGAVHTRGLDFACD
(SEQ ID NO: 98)
CD8a TM (72 nt)
Atctacatctgggcgcccttggccgggacttgtggggtccttctcctgtcactggttatcaccctttactgc (SEQ ID NO: 99)
CD8a TM amino acid sequence
IYIWAPLAGTCGVLLLSLVITLYC (SEQ ID NO: 100)
4-1BB co-stimulatory region (126 nt)
Aaacggggcagaaagaaactcctgtatatattcaaacaaccatttatgagaccagtacaaactactcaagaggaagatggctgtag
ctgccgatttccagaagaagaagaaggaggatgtgaactg (SEQ ID NO: 101)
CD3z cytoplasmic signaling domain (336 nt)
Agagtgaagttcagcaggagcgcagacgcccccgcgtaccagcagggccagaaccagctctataacgagctcaatctaggacg
aagagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagggg
caaggggcacgatggcctttaccagggtctcagtacagccaccaaggacacctacgacgcccttcacatgcaggccctgccccct
cgctaa (SEQ ID NO: 102)
TB19-L1 (2235 nt)
ATGGCCTTACCAGTGACCGCCTTGCTCCTGCCGCTGGCCTTGCTGCTCCACGCCG
CCAGGCCGgacatccagatgacccagtccccctcctccctgtccgcctccgtgggcgaccgggtgaccatcacctgccgg
gcctcccagggcatctccaactacctgaactggtaccagcagaagcccggcaaggcccccaagcccctgatctactacacctcca
acctgcagtccggcgtgccctcccggttctccggctccggctccggcaccgactacaccctgaccatctcctccctgcagcccgag
gacttcgccacctactactgcatgggccagaccatctcctcctacaccttcggccagggcaccaagctggagatcaagggtggcg
gtggctcgggcggtggtgggtcgggtggcggcggatctgaggtgcagctggtggagtccggcggcggcctggtgcagcccgg
cggctccctgcggctgtcctgcgccgcctccggcttcaccttctccaacttcgacatggcctgggtgcggcaggcccccggcaag
ggcctggtgtgggtgtcctccatcaccaccggcgccgaccacgccatctacgccgactccgtgaagggccggttcaccatctccc
gggacaacgccaagaacaccctgtacctgcagatgaactccctgcgggccgaggacaccgccgtgtactactgcgtgcggcacg
gctactacgacggctaccacctgttcgactactggggccagggcaccctggtgaccgtgtcctccGGAGGTGGTGGAT
CCgacatccagatgacacagactacatcctccctgtctgcctctctgggagacagagtcaccatcagttgcagggcaagtcagga
cattagtaaatatttaaattggtatcagcagaaaccagatggaactgttaaactcctgattaccatacatcaagattacactcag gagt
cccatcaaggttcagtggcagtgggtctggaacagattattctctcaccattagcaacctggagcaagaagatattgccacttactttt
gccaacagggtaatacgcttccgtacacgttcggaggggggaccaagctggagatcacaggtggcggtggctcgggcggtggt
gggtcgggtggcggcggatctgaggtgaaactgcaggagtcaggacctggcctggtggcgccctcacagagcctgtccgtcaca
tgcactgtctcaggggtctcattacccgactatggtgtaagctggattcgccagcctccacgaaagggtctggagtggctgggagta
atatggggtagtgaaaccacatactataattcagctctcaaatccagactgaccatcatcaaggacaactccaagagccaagttttctt
aaaaatgaacagtctgcaaactgatgacacagccatttactactgtgccaaacattattactacggtggtagctat gctatggactact
ggggccaaggaacctcagtcaccgtctcctcattcgtgccggtcttcctgccagcgaagcccaccacgacgccagcgccgcgac
caccaacaccggcgcccaccatcgcgtcgcagcccctgtccctgcgcccagaggcgtgccggccagcggggggggcgcagt
gcacacgagggggctggacttcgcctgtgatatctacatctgggcgcccttggccgggacttgtggggtccttctcctgtcactggtt
atcaccctttactgcaaacggggcagaaagaaactcctgtatatattcaaacaaccatttatgagaccagtacaaactactcaagagg
aagatggctgtagctgccgatttccagaagaagaagaaggaggatgtgaactgAgagtgaagttcagcaggagcgcagacgcc
cccgcgtaccagcagggccagaaccagctctataacgagctcaatctaggacgaagagaggagtacgatgtttt ggacaagaga
cgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctc
agtacagccaccaaggacacctacgacgcccttcacatgcaggccctgccccctegctaa (SEQ ID NO: 103)
TB19-L1 amino acid sequence
MALPVTALLLPLALLLHAARPDIQMTQSPSSLSASVGDRVTITCRASQGISNYLN
WYQQKPGKAPKPLIYYTSNLQSGVPSRFSGSGSGTDYTLTISSLQPEDFATYYCM
GQTISSYTFGQGTKLEIKGGGGSGGGGSGGGGSEVQLVESGGGLVQPGGSLRLS
CAASGFTFSNFDMAWVRQAPGKGLVWVSSITTGADHAIYADSVKGRFTISRDNA
KNTLYLQMNSLRAEDTAVYYCVRHGYYDGYHLFDYWGQGTLVTVSSGGGGSD
IQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIYYCAK
HYYYGGSYAMDYWGQGTSVTVSSFVPVFLPAKPTTTPAPRPPTPAPTIASQPLSL
RPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAY
SEIGMKGERRRGKGHDGLYQGLSTATKDTYDALHMQALPPR (SEQ ID NO: 104)
TN-B20-19-L2 (TB19-L2)
CD8a SP (63 nt)
ATGGCCTTACCAGTGACCGCCTTGCTCCTGCCGCTGGCCTTGCTGCTCCACGCCG
CCAGGCCG (SEQ ID NO: 105)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 106)
Linker-1 (45 nt)
ggtggcggtggctcgggcggtggtgggtcgggtggcggcggatct (SEQ ID NO: 107)
Linker-1 amino acid sequence
GGGGSGGGGSGGGGS (SEQ ID NO: 108)
B20 V_{L} (321 nt)
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctceggcaccgactacaccctgaccatctcctccctgcagcccgaggacttcgccacc
tactactgcatgggccagaccatctcctcctacaccttcggccagggcaccaagctggagatcaag (SEQ ID NO: 109)
Linker-2 (15 nt)
GGAGGTGGTGGATCC (SEQ ID NO: 110)
Linker-2 amino acid sequence:
GGGGS (SEQ ID NO: 111)
FMC63 V_{L} (321 nt)
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 112)
Linker-3 (45 nt)
Ggtggcggtggctcgggcggtggtgggtcgggtggcggcggatct (SEQ ID NO: 113)
Linker-3 amino acid sequence
GGGGSGGGGSGGGGS (SEQ ID NO: 114)
FMC63 V_{H} (360 nt)
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 115)
CD8a hinge (165 nt)
Ttcgtgccggtcttcctgccagcgaagcccaccacgacgccagcgccgcgaccaccaacaccggcgcccaccatcgcgtcgca
gcccctgtccctgcgcccagaggcgtgccggccagcggcggggggcgcagtgcacacgagggggctggacttcgcctgtgat
(SEQ ID NO: 116)
CD8a TM (72 nt)
Atctacatctgggcgcccttggccgggacttgtggggtccttctcctgtcactggttatcaccctttactgc (SEQ ID NO: 117)
4-1BB co-stimulatory region (126 nt)
Aaacggggcagaaagaaactcctgtatatattcaaacaaccatttatgagaccagtacaaactactcaagaggaagatggctgtag
ctgccgatttccagaagaagaagaaggaggatgtgaactg (SEQ ID NO: 118)
CD3z cytoplasmic signaling domain (336 nt)
Agagtgaagttcagcaggagcgcagacgcccccgcgtaccagcagggccagaaccagctctataacgagctcaatctaggacg
aagagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagggg
caaggggcacgatggcctttaccagggtctcagtacagccaccaaggacacctacgacgcccttcacatgcaggccctgccccct
cgctaa (SEQ ID NO: 119)
TB19-L2 (2235 nt)
ATGGCCTTACCAGTGACCGCCTTGCTCCTGCCGCTGGCCTTGCTGCTCCACGCCG
CCAGGCCGgaggtgcagctggtggagtccggcggcggcctggtgcagcccggcggctccctgcggctgtcctgcgccgc
ctccggcttcaccttctccaacttcgacatggcctgggtgcggcaggcccccggcaagggcctggtgtgggtgtcctccatcacca
ccggcgccgaccacgccatctacgccgactccgtgaagggccggttcaccatctcccgggacaacgccaagaacaccctgtacc
tgcagatgaactccctgcgggccgaggacaccgccgtgtactactgcgtgcggcacggctactacgacggctaccacctgttcga
ctactggggccagggcaccctggtgaccgtgtcctccggtggcggtggctcgggcggtggtgggtcgggtggcggcggatctga
catccagatgacccagtccccctcctccctgtccgcctccgtgggcgaccgggtgaccatcacctgccgggcctcccagggcatc
tccaactacctgaactggtaccagcagaagcccggcaaggcccccaagcccctgatctactacacctccaacctgcagtccggcg
tgccctcccggttctccggctccggctccggcaccgactacaccctgaccatctcctccctgcagcccgaggacttcgccacctact
actgcatgggccagaccatctcctcctacaccttcggccagggcaccaagctggagatcaagGGAGGTGGTGGATC
Cgacatccagatgacacagactacatcctccctgtctgcctctctgggagacagagtcaccatcagttgcagggcaagtcaggac
attagtaaatatttaaattggtatcagcagaaaccagatggaactgttaaactcctgatctaccatacatcaagattacactcaggagtc
ccatcaaggttcagtggcagtgggtctggaacagattattctctcaccattagcaacctggagcaagaagatattgccacttacttttg
ccaacagggtaatacgcttccgtacacgttcggaggggggaccaagctggagatcacaggtggcggtggctcgggcggtggtg
ggtcgggtggcggcggatctgaggtgaaactgcaggagtcaggacctggcctggtggcgccctcacagagcctgtccgtcacat
gcactgtctcaggggtctcattacccgactatggtgtaagctggattcgccagcctccacgaaagggtctggagtggctgggagta
atatggggtagtgaaaccacatactataattcagctctcaaatccagactgaccatcatcaaggacaactccaagagccaagttttctt
aaaaatgaacagtctgcaaactgatgacacagccatttactactgtgccaaacattattactacggtggtagctatgctatggactact
ggggccaaggaacctcagtcaccgtctcctcattcgtgccggtcttcctgccagcgaagcccaccacgacgccagcgccgcgac
caccaacaccggcgcccaccatcgcgtcgcagcccctgtccctgcgcccagaggcgtgccggccagcggcggggggcgcagt
gcacacgagggggctggacttcgcctgtgatatctacatctgggcgcccttggccgggacttgtggggtccttctcctgtcactggtt
atcaccctttactgcaaacggggcagaaagaaactcctgtatatattcaaacaaccatttatgagaccagtacaaactactcaagagg
aagatggctgtagctgccgatttccagaagaagaagaaggaggatgtgaactgAgagtgaagttcagcaggagcgcagacgcc
cccgcgtaccagcagggccagaaccagctctataacgagctcaatctaggacgaagagaggagtacgatgttttggacaagaga
cgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctc
agtacagccaccaaggacacctacgacgcccttcacatgcaggccctgccccctcgctaa (SEQ ID NO: 120)
TB19-L2 amino acid sequence
MALPVTALLLPLALLLHAARPEVQLVESGGGLVQPGGSLRLSCAASGFTFSNFD
MAWVRQAPGKGLVWVSSITTGADHAIYADSVKGRFTISRDNAKNTLYLQMNSL
RAEDTAVYYCVRHGYYDGYHLFDYWGQGTLVTVSSGGGGSGGGGSGGGGSDI
QMTQSPSSLSASVGDRVTITCRASQGISNYLNWYQQKPGKAPKPLIYYTSNLQSG
VPSRFSGSGSGTDYTLTISSLQPEDFATYYCMGQTISSYTFGQGTKLEIKGGGGS
DIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGG
GSGGGGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAIYYCA
KHYYYGGSYAMDYWGQGTSVTVSSFVPVFLPAKPTTTPAPRPPTPAPTIASQPLS
LRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEA
YSEIGMKGERRRGKGHDGLYQGLSTATKDTYDALHMQALPPR (SEQ ID
NO: 121)
TN-B20-19-L3 (TB19-L3)
CD8a SP (63 nt)
ATGGCCTTACCAGTGACCGCCTTGCTCCTGCCGCTGGCCTTGCTGCTCCACGCCG
CCAGGCCG (SEQ ID NO: 122)
B20 V_{H}
Gaggtgcagctggtggagtccggcggcggcctggtgcagcccggcggctccctgcggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 123)
Linker-1 (45 nt)
ggtggcggtggctcgggcggtggtgggtcgggtggcggcggatct (SEQ ID NO: 124)
Linker-1 amino acid sequence:
GGGGSGGGGSGGGGS (SEQ ID NO: 125)
B20 V_{L} (321 nt)
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 126)
Linker-2 (15 nt)
GGAGGTGGTGGATCC (SEQ ID NO: 127)
Linker-2 amino acid sequence
GGGGS (SEQ ID NO: 128)
FMC63 V_{H} (360 nt)
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 129)
Linker-3 (45 nt)
Ggtggcggtggctcgggcggtggtgggtcgggtggcggcggatct (SEQ ID NO: 130)
Linker-3 amino acid sequence
GGGGSGGGGSGGGGS (SEQ ID NO: 131)
FMC63 V_{L} (321 nt)
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgattaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 132)
CD8a hinge (165 nt)
Ttcgtgccggtcttcctgccagcgaagcccaccacgacgccagcgccgcgaccaccaacaccggcgcccaccatcgcgtcgca
gcccctgtccctgcgcccagaggcgtgccggccagcgggcgggggcgcagtgcacacgagggggctggacttcgcctgtgat
(SEQ ID NO: 133)
CD8a TM (72 nt)
Atctacatctgggcgcccttggccgggacttgtggggtccttctcctgtcactggttatcaccctttactgc (SEQ ID NO: 134)
4-1BB co-stimulatory region (126 nt)
Aaacggggcagaaagaaactcctgtatatattcaaacaaccatttatgagaccagtacaaactactcaagaggaagatggctgtag
ctgccgatttccagaagaagaagaaggaggatgtgaactg (SEQ ID NO: 135)
CD3z cytoplasmic signaling domain (336 nt)
Agagtgaagttcagcaggagcgcagacgcccccgcgtaccagcagggccagaaccagctctataacgagctcaatctaggacg
aagagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagggg
caaggggcacgatggcctttaccagggtctcagtacagccaccaaggacacctacgacgcccttcacatgcaggccctgccccct
cgctaa (SEQ ID NO: 136)
TB19-L3 (2235nt)
ATGGCCTTACCAGTGACCGCCTTGCTCCTGCCGCTGGCCTTGCTGCTCCACGCCG
CCAGGCCGgaggtgcagctggtggagtccggcggcggcctggtgcagcccggggctccctgcggctgtcctgcgccgc
ctccggcttcaccttctccaacttcgacatggcctgggtgcggcaggcccccggcaagggcctggtgtgggtgtcctccatcacca
ccggcgccgaccacgccatctacgccgactccgtgaagggccggttcaccatctcccgggacaacgccaagaacaccctgtacc
tgcagatgaactccctgcgggccgaggacaccgccgtgtactactgcgtgcggcacggctactacgacggctaccacctgttcga
ctactggggccagggcaccctggtgaccgtgtcctccggtggcggtggctcgggcggtggtgggtcgggtggcggcggatctga
catccagatgacccagtccccctcctccctgtccgcctccgtgggcgaccgggtgaccatcacctgccgggcctcccagggcatc
tccaactacctgaactggtaccagcagaagcccggcaaggcccccaagcccctgatctactacacctccaacctgcagtccggcg
tgccctcccggttctccggctccggctccggcaccgactacaccctgaccatctcctccctgcagcccgaggacttcgccacctact
actgcatgggccagaccatctcctcctacaccttcggccagggcaccaagctggagatcaagGGAGGTGGTGGATC
Cgaggtgaaactgcaggagtcaggacctggcctggtggcgccctcacagagcctgtccgtcacatgcactgtctcaggggtctca
ttacccgactatggtgtaagctggattcgccagcctccacgaaagggtctggagtggctgggagtaatatggggtagtgaaaccac
atactataattcagctctcaaatccagactgaccatcatcaaggacaactccaagagccaagttttcttaaaaatgaacagtctgcaaa
ctgatgacacagccatttactactgtgccaaacattattactacggtggtagctatgctatggactactggggccaaggaacctcagt
caccgtctcctcaggtggcggtggctcgggcggtggtgggtcgggtggcggcggatctgacatccagatgacacagactacatcc
tccctgtctgcctctctgggagacagagtcaccatcagttgcagggcaagtcaggacattagtaaatatttaaattggtatcagcaga
aaccagatggaactgttaaactcctgatctaccatacatcaagattacactcaggagtcccatcaaggttcagtggcagtgggtctgg
aacagattattctctcaccattagcaacctggagcaagaagatattgccacttacttttgccaacagggtaatacgcttccgtacacgtt
cggaggggggaccaagctggagatcacattcgtgccggtcttcctgccagcgaagcccaccacgacgccagcgccgcgaccac
caacaccggcgcccaccatcgcgtcgcagcccctgtccctgcgcccagaggcgtgccggccagcggcggggggcgcagtgca
cacgagggggctggacttcgcctgtgatatctacatctgggcgcccttggccgggacttgtggggtccttctcctgtcactggttatc
accctttactgcaaacggggcagaaagaaactcctgtatatattcaaacaaccatttatgagaccagtacaaactactcaagaggaa
gatggctgtagctgccgatttccagaagaagaagaaggaggatgtgaactgAgagtgaagttcagcaggagcgcagacgcccc
cgcgtaccagcagggccagaaccagctctataacgagctcaatctaggacgaagagaggagtacgatgttttggacaagagacgt
ggccgggaccctgagatggggggaaagccgagaaggaagaaccctcaggaaggcctgtacaatgaactgcagaaagataaga
tggcggaggcctacagtgagattgggatgaaaggcgagcgccggaggggcaaggggcacgatggcctttaccagggtctcagt
acagccaccaaggacacctacgacgcccttcacatgcaggccctgccccctcgctaa (SEQ ID NO: 137)
TB19-L3 amino acid sequence
MALPVTALLLPLALLLHAARPEVQLVESGGGLVQPGGSLRLSCAASGFTFSNFD
MAWVRQAPGKGLVWVSSITTGADHAIYADSVKGRFTISRDNAKNTLYLQMNSL
RAEDTAVYYCVRHGYYDGYHLFDYWGQGTLVTVSSGGGGSGGGGSGGGGSDI
QMTQSPSSLSASVGDRVTITCRASQGISNYLNWYQQKPGKAPKPLIYYTSNLQSG
VPSRFSGSGSGTDYTLTISSLQPEDFATYYCMGQTISSYTFGQGTKLEIKGGGGS
EVKLQESGPGLVAPSQSLSVTCTVSGVSLPDYGVSWIRQPPRKGLEWLGVIWGS
ETTYYNSALKSRLTIIKDNSKSQVFLKMNSLQTDDTAIYYCAKHYYYGGSYAMD
YWGQGTSVTVSSGGGGSGGGGSGGGGSDIQMTQTTSSLSASLGDRVTISCRASQ
DISKYLNWYQQKPDGTVKLLIYHTSRLHSGVPSRFSGSGSGTDYSLTISNLEQED
IATYFCQQGNTLPYTFGGGTKLEITFVPVFLPAKPTTTPAPRPPTPAPTIASQPLS
LRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEA
YSEIGMKGERRRGKGHDGLYQGLSTATKDTYDALHMQALPPR (SEQ ID
NO: 138)
TN-B20-19-L4 (TB19-L4)
CD8a SP (63 nt)
ATGGCCTTACCAGTGACCGCCTTGCTCCTGCCGCTGGCCTTGCTGCTCCACGCCG
CCAGGCCG (SEQ ID NO: 139)
B20 V_{L} (321 nt)
Gacatccagatgacccagtccccctcctccctgtccgcctccgtgggcgaccgggtgaccatcacctgccgggcctcccagggc
atctccaactacctgaactggtaccagcagaagcccggcaaggcccccaagcccctgatctactacacctccaacctgcagtccg
gcgtgccctcccggttctccggctccggctccggcaccgactacaccctgaccatctcctccctgcagcccgaggacttcgccacc
tactactgcatgggccagaccatctcctcctacaccttcggccagggcaccaagctggagatcaag (SEQ ID NO: 140)
Linker-1 (45nt)
ggtggcggtggctcgggcggtgggggtcgggtggcggcggatct (SEQ ID NO: 141)
Linker-1 amino acid sequence:
GGGGSGGGGSGGGGS (SEQ ID NO: 142)
B20 V_{H}
Gaggtgcagctggtggagtccggcggcggcctggtgcagcccggcggctccctgeggctgtcctgcgccgcctccggcttcac
cttctccaacttcgacatggcctgggtgcggcaggcccccggcaagggcctggtgtgggtgtcctccatcaccaccggcgccgac
cacgccatctacgccgactccgtgaagggccggttcaccatctcccgggacaacgccaagaacaccctgtacctgcagatgaact
ccctgcgggccgaggacaccgccgtgtactactgcgtgcggcacggctactacgacggctaccacctgttcgactactggggcca
gggcaccctggtgaccgtgtcctcc (SEQ ID NO: 143)
Linker-2 (15 nt)
GGAGGTGGTGGATCC (SEQ ID NO: 144)
Linker-2 amino acid sequence
GGGGS (SEQ ID NO: 145)
FMC63 V_{H} (360 nt)
Gaggtgaaactgcaggagtcaggacctggcctggtggcgccctcacagagcctgtccgtcacatgcactgtctcaggggtctcat
tacccgactatggtgtaagctggattcgccagcctccacgaaagggtctggagtggctgggagtaatatggggtagtgaaaccaca
tactataattcagctctcaaatccagactgaccatcatcaaggacaactccaagagccaagttttcttaaaaatgaacagtctgcaaac
tgatgacacagccatttactactgtgccaaacattattactacggtggtagctatgctatggactactggggccaaggaacctcagtc
accgtctcctca (SEQ ID NO: 146)
Linker-3 (45 nt)
Ggtggcggtggctcgggcggtggtgggtcgggtggcggcggatct (SEQ ID NO: 147)
Linker-3 amino acid sequence
GGGGSGGGGSGGGGS (SEQ ID NO: 148)
FMC63 V_{L} (321 nt)
Gacatccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggagtccc
atcaaggttcagtggcagtgggtctggaacagattattctctcaccattagcaacctggagcaagaagatattgccacttacttttgcc
aacagggtaatacgcttccgtacacgttcggaggggggaccaagctggagatcaca (SEQ ID NO: 149)
CD8a hinge (165 nt)
Ttcgtgccggtcttcctgccagcgaagcccaccacgacgccagcgccgcgaccaccaacaccggcgcccaccatcgcgtcgca
gcccctgtccctgcgcccagaggcgtgccggccagcggcggggggcgcagtgcacacgagggggctggacttcgcctgtgat
(SEQ ID NO: 150)
CD8a TM (72 nt)
Atctacatctgggcgcccttggccgggacttgtggggtccttctcctgtcactggttatcaccctttactgc (SEQ ID NO: 151)
4-1BB co-stimulatory region (126 nt)
Aaacggggcagaaagaaactcctgtatatattcaaacaaccatttatgagaccagtacaaactactcaagaggaagatggctgtag
ctgccgatttccagaagaagaagaaggaggatgtgaactg (SEQ ID NO: 152)
CD3z cytoplasmic signaling domain (336 nt)
Agagtgaagttcagcaggagcgcagacgcccccgcgtaccagcagggccagaaccagctctataacgagctcaatctaggacg
aagagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagggg
caaggggcacgatggcctttaccagggtctcagtacagccaccaaggacacctacgacgcccttcacatgcaggccctgccccct
cgctaa (SEQ ID NO: 153)
TB19-L4 (2235 nt)
ATGGCCTTACCAGTGACCGCCTTGCTCCTGCCGCTGGCCTTGCTGCTCCACGCCG
CCAGGCCGGacatccagatgacccagtccccctcctccctgtccgcctccgtgggcgaccgggtgaccatcacctgccgg
gcctcccagggcatctccaactacctgaactggtaccagcagaagcccggcaaggcccccaagcccctgatctactacacctcca
acctgcagtccggcgtgccctcccggttctccggctccggctccggcaccgactacaccctgaccatctcctccctgcagcccgag
gacttcgccacctactactgcatgggccagaccatctcctcctacaccttcggccagggcaccaagctggagatcaagggtggcg
gtggctcgggcggtggtgggtcgggtggcggcggatctgaggtgcagctggtggagtccggcggcggcctggtgcagcccgg
cggctccctgcggctgtcctgcgccgcctccggcttcaccttctccaacttcgacatggcctgggtgcggcaggcccccggcaag
ggcctggtgtgggtgtcctccatcaccaccggcgccgaccacgccatctacgccgactccgtgaagggccggttcaccatctccc
gggacaacgccaagaacaccctgtacctgcagatgaactccctgcgggccgaggacaccgccgtgtactactgcgtgcggcacg
gctactacgacggctaccacctgttcgactactggggccagggcaccctggtgaccgtgtcctccGGAGGTGGTGGAT
CCgaggtgaaactgcaggagtcaggacctggcctggtggcgccctcacagagcctgtccgtcacatgcactgtctcaggggtct
cattacccgactatggtgtaagctggattcgccagcctccacgaaagggtctggagtggctgggagtaatatggggtagtgaaacc
acatactataattcagctctcaaatccagactgaccatcatcaaggacaactccaagagccaagttttcttaaaaatgaacagtctgca
aactgatgacacagccatttactactgtgccaaacattattactacggtggtagctatgctatggactactggggccaaggaacctca
gtcaccgtctcctcaggggcggtggctcgggcggtggtgggtcgggtggcggcggatctgacatccagatgacacagactacat
cctccctgtctgcctctctgggagacagagtcaccatcagttgcagggcaagtcaggacattagtaaatatttaaattggtatcagca
gaaaccagatggaactgttaaactcctgatctaccatacatcaagattacactcaggagtcccatcaaggttcagtggcagtgggtct
ggaacagattattctctcaccattagcaacctggagcaagaagatattgccacttacttttgccaacagggtaatacgcttccgtacac
gttcggaggggggaccaagctggagatcacattcgtgccggtcttcctgccagcgaagcccaccacgacgccagcgccgcgac
caccaacaccggcgcccaccatcgcgtcgcagcccctgtccctgcgcccagaggcgtgccggccagcggcggggggcgcagt
gcacacgagggggctggacttcgcctgtgatatctacatctgggcgcccttggccgggacttgtggggtccttctcctgtcactggtt
atcaccctttactgcaaacggggcagaaagaaactcctgtatatattcaaacaaccatttatgagaccagtacaaactactcaagagg
aagatggctgtagctgccgatttccagaagaagaagaaggaggatgtgaactgAgagtgaagttcagcaggagcgcagacgcc
cccgcgtaccagcagggccagaaccagctctataacgagctcaatctaggacgaagagaggagtacgatgttttggacaagaga
cgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctc
agtacagccaccaaggacacctacgacgcccttcacatgcaggccctgccccctegctaa (SEQ ID NO: 154)
TB19-L4 amino acid sequence
MALPVTALLLPLALLLHAARPDIQMTQSPSSLSASVGDRVTITCRASQGISNYLN
WYQQKPGKAPKPLIYYTSNLQSGVPSRFSGSGSGTDYTLTISSLQPEDFATYYCM
GQTISSYTFGQGTKLEIKGGGGSGGGGSGGGGSEVQLVESGGGLVQPGGSLRLS
CAASGFTFSNFDMAWVRQAPGKGLVWVSSITTGADHAIYADSVKGRFTISRDNA
KNTLYLQMNSLRAEDTAVYYCVRHGYYDGYHLFDYWGQGTLVTVSSGGGGSE
VKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDY
WGQGTSVTVSSGGGGSGGGGSGGGGSDIQMTQTTSSLSASLGDRVTISCRASQD
ISKYLNWYQQKPDGTVKLLIYHTSRLHSGVPSRFSGSGSGTDYSLTISNLEQEDI
ATYFCQQGNTLPYTFGGGTKLEITFVPVFLPAKPTTTPAPRPPTPAPTIASQPLSL
RPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAY
SEIGMKGERRRGKGHDGLYQGLSTATKDTYDALHMQALPPR (SEQ ID NO: 155)

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A bispecific chimeric antigen receptor (CAR), comprising:
(i) an anti-BCMA antigen-binding region which comprises a light chain variable region (V_{L}1) and a heavy chain variable region (V_{H}1), wherein V_{L}1 comprises three complementarity determining regions (CDRs), CDR1, CDR2 and CDR3, having amino acid sequences about 80% to about 100% identical to the amino acid sequences set forth in SEQ ID NO: 2, SEQ ID NO: 4, SEQ ID NO: 6, respectively, and wherein V_{H}1 comprises three CDRs, CDR1, CDR2 and CDR3, having amino acid sequences about 80% to about 100% identical to the amino acid sequences set forth in SEQ ID NO: 9, SEQ ID NO: 11, SEQ ID NO: 13, respectively; and
(ii) an anti-CD19 antigen-binding region which comprises a light chain variable region (V_{L}2) and a heavy chain variable region (V_{H}2), wherein V_{L}2 comprises three complementarity determining regions (CDRs), CDR1, CDR2 and CDR3, having amino acid sequences about 80% to about 100% identical to the amino acid sequences set forth in SEQ ID NO: 15, SEQ ID NO: 16, SEQ ID NO: 17, respectively, and wherein V_{H}2 comprises three CDRs, CDR1, CDR2 and CDR3, having amino acid sequences about 80% to about 100% identical to the amino acid sequences set forth in SEQ ID NO: 18, SEQ ID NO: 19, SEQ ID NO: 20, respectively.

2. The bispecific CAR of claim 1, wherein V_{L}1 is located at the N-terminus of V_{H}1.

3. The bispecific CAR of claim 1, wherein V_{L}2 is located at the N-terminus of V_{H}2.

4. The bispecific CAR of claim 1, wherein V_{H}1 is located at the N-terminus of V_{L}I.

5. The bispecific CAR of claim 1, wherein V_{H}2 is located at the N-terminus of V_{L}2.

6. The bispecific CAR of claim 1, wherein V_{L}1 and V_{H}1 have amino acid sequences about 80% to about 100% identical to amino acid sequences set forth in SEQ ID NO: 24 and SEQ ID NO: 28, respectively.

7. The bispecific CAR of claim 1, wherein V_{L}2 and V_{H}2 have amino acid sequences about 80% to about 100% identical to amino acid sequences set forth in SEQ ID NO: 32 and SEQ ID NO: 36, respectively.

8. The bispecific CAR of claim 1, wherein the anti-BCMA antigen-binding region is a single-chain variable fragment (scFv) that specifically binds BCMA, and wherein the anti-CD19 antigen-binding region is a scFv that specifically binds CD19.

9. The bispecific CAR of claim 1, wherein the bispecific CAR further comprises one or more of the following:
(a) a signal peptide,
(b) a hinge region,
(c) a transmembrane domain,
(d) a co-stimulatory region, and
(e) a cytoplasmic signaling domain.

10. The bispecific CAR of claim 9, wherein the co-stimulatory region comprises a co-stimulatory region of 4-1BB (CD137), CD28, OX40, CD2, CD7, CD27, CD30, CD40, CD70, CD134, PD1, Dap10, CDS, ICAM-1, LFA-1 (CD11a/CD18), ICOS (CD278), NKG2D, GITR, TLR2, or combinations thereof.

11. The bispecific CAR of claim 9, wherein the cytoplasmic signaling domain comprises a cytoplasmic signaling domain of CD3ζ.

12. The bispecific CAR of claim 9, wherein the hinge region comprises a hinge region of IgG4, CD8, CD28, CD137, or combinations thereof.

13. The bispecific CAR of claim 9, wherein the transmembrane domain comprises a transmembrane domain of CD8, CD28, CD3ε, CD45, CD4, CD5, CD9, CD16, CD22, CD33, CD37, CD64, CD80, CD86, CD134, CD137, CD154, or combinations thereof.

14. The bispecific CAR of claim 9, comprising an amino acid sequence about 80% to about 100% identical to the amino acid sequence set forth in SEQ ID NO:46, SEQ ID NO:60, SEQ ID NO:74, SEQ ID NO:88, SEQ ID NO:104, SEQ ID NO:121, SEQ ID NO:138, or SEQ ID NO:155.

15. An immune cell expressing the bispecific CAR of claim 1.

16. The immune cell of claim 15, wherein the immune cell is a T cell or a natural killer (NK) cell.

17. A nucleic acid encoding the bispecific CAR of claim 1.

18. A method of treating an autoimmune disorder, the method comprising administering the immune cell of claim 15 to a subject in need thereof.

19. The method of claim 18, wherein the autoimmune disorder is systemic lupus erythematosus (SLE), systemic sclerosis, inflammatory myopathy, systemic scleroderma, multiple sclerosis, myasthenia gravis, a myositis autoantibody-driven disease, or neuromyelitis optica.

20. The method of claim 19, wherein the inflammatory myopathy is polymyositis, dermatomyositis, or inclusion-body myositis.

21. The method of claim 19, wherein the subject has lupus nephritis.

22. The method of claim 18, wherein the immune cell is allogeneic or autologous.
